(19) United States
(12) Patent Application Publication
(10) Pub. No.: US 2020/0010502 A1
(43) Pub. Date: Jan. 9, 2020

(54) SYNTHESIS OF MULTIPHOSPHORYLATED PEPTIDES

(71) Applicant: YISSUM RESEARCH DEVELOPMENT COMPANY OF THE HEBREW UNIVERSITY OF JERUSALEM LTD., Jerusalem (IL)

(72) Inventors: Assaf FRIEDLER, Mevasseret Zion (IM); Samara simha reddy MAMDI, Horby (SE); Mattan HUREVICH, Jerusalem (IL)

(21) Appl. No.: 16/502,616

(22) Filed: Jul. 3, 2019

Related U.S. Application Data
(60) Provisional application No. 62/694,108, filed on Jul. 5, 2018.

(51) Int. Cl. C07K 1/04; C07K 7/08; C07K 7/06

(52) U.S. Cl. CPC C07K 1/04; C07K 7/06; C07K 7/08

(57) ABSTRACT

The present invention relates to a new approach for the synthesis of multiphosphorylated peptides. Specifically, the present invention provides a process, which enables the synthesis of multiphosphorylated peptides with up to seven phosphorylated Serine (pSer) and Threonine (pThr) residues, including such residues that are close in sequence.

### BACKGROUND OF THE INVENTION

Synthetic multiphosphorylated peptides are essential for studying the mechanism of action and regulation of multiphosphorylated proteins as well as the correlation between the phosphorylation pattern and the biological function of proteins. Specifically, phosphorylation and dephosphorylation play a vital role in regulation of numerous cellular processes (1, 2). Diseases such as Cancer (3, 4) and diabetes (5, 6) are associated with impaired phosphorylation pathways. Chemical peptide synthesis is the only way to ensure homogeneous site-specific phosphorylation at the required residues within the sequence. Thus, synthetic phosphopeptides derived from phosphoprotein sequences are crucial for studying the mechanism of action and regulation of a large number of proteins, including disease-related ones and for dissecting the specific role of each phosphorylated residue (7-10). Many cell-cycle regulatory proteins undergo multiple phosphorylations at multiple residues, leading to changes in their biochemical properties and biological functions (11). The number of phosphorylation sites observed in proteins varies from 1 to over 100 (12). Multisite phosphorylation in proteins is a common mechanism for increasing their level of regulation (13).

Homogeneous, synthetic multiphosphorylated peptides are key tools for understanding these regulatory mechanisms as they allow for a systematic screening of combinations of multi phosphorylation patterns. Multiphosphorylated peptides are useful for evaluating the correlation between the phosphorylation pattern and the biological function of proteins.

While mono phosphorylated peptides can be efficiently accessed using protected phosphorylated amino acids and a single fluorenylmethyloxycarbonyl solid phase peptide synthesis (Fmoc-SPPS) protocol, the synthesis of multi phosphorylated peptides is limited because the coupling efficiency depends on the number and pattern of the phosphorylated amino acids (pAAs). Several approaches have been developed for the synthesis of phosphopeptides, including the global phosphorylation approach and the building block approach (14-22).

Despite the high importance of multiphosphorylated peptides, their synthesis is extremely difficult. Conventional Fmoc-SPPS of phosphorylated peptides with up to six phosphorylated serine residues was previously reported (35, 36). Both examples describe the synthesis of a multiple phosphorylated peptide containing only phosphorylated Ser residues. In addition, the phosphorylated Ser residues are separated by at least one non-phosphorylated residue. In these reports, large excess of amino acids and multiple repetitive coupling cycles were used for increasing the coupling efficiency. The synthesis of phosphorylated peptides using standard Fmoc-SPPS protocols suffer from low coupling efficiency of all three phosphorylated amino acids, Ser, Thr and Tyr (Tyrosine). The coupling of Fmoc-Thr(HPO₃Bzl)-OH proved extremely challenging even when the most reactive coupling reagents were used (37).

Microwave (MW) assisted SPPS is a useful tool for the synthesis of peptides with difficult sequences that are challenging and sometimes impossible to synthesize using conventional SPPS conditions (38-41). The first MW assisted SPPS of a 15-mer peptide bearing a single pSer utilizing the Fmoc-Ser(HPO₃Bzl)-OH building block resulted in increase of the coupling efficiency (40). However, it was suggested that the mono-benzyl protected phosphorylated amino acids are not compatible with the MW assisted Fmoc deprotection conditions. The automated synthesis of β-catenin derived peptides bearing up to three pAAs was performed using a combination of MW assisted and conventional Fmoc-SPPS protocols (42). The pAAs and their adjacent amino acids were coupled using 5 equivalents of amino acids using MW at 72° C. for 15 min employing HBTU/DIEA activation system. The β-catenin peptide synthesized has two pSer residues and only one pThr that are not in close proximity and are separated from each other by three non-phosphorylated residues.

The synthesis of multi phosphorylated peptide with up to seven phosphorylations was reported employing the hazardous Boc-SPPS method. The di-benzyl protecting group removal and the peptide release from the resin were performed using hydrogenolysis in presence of palladium and platinum. This hydrogenolysis step took 4 days before cleavage could be performed. This reported procedure is time consuming and employs very harsh and inconvenient heterogeneous solid phase protocols, which make this strategy highly inaccessible (43-45).

Rhodopsin (Rho) is a light sensitive G protein coupled receptor that enables vision and involves in large number of regulatory mechanisms (46). The cellular C-terminal region of the Rho, of residues 330 to 348 (DDEASTTVSKTETSQVAPA) is highly phosphorylated as indicated by phosphorylated residues underlined in the sequence (47, 48). This peptide contains a combination of three pSer and four pThr amino acid residues. Moreover, it contains two regions with neighboring phosphorylated residues that are not separated by non-phosphorylated residues. Fmoc SPPS of peptides derived from Rho 330-348 is extremely challenging as it requires the introduction of up to four Fmoc-Thr(HPO₃Bzl)-OH residues and the coupling of contiguous phosphorylated amino acids. The Fmoc-SPPS of phosphorylated peptides bearing multiple phosphorylated threonine residues with adjacent phosphorylated amino acids was never reported for any target.

Several attempt to synthesize Rho derived peptides using previously reported MW assisted Fmoc-SPPS protocols resulted in a sharp decrease in yield and purity after the introduction of the first two protected phosphorylated amino acids residues.

Thus, there is an unmet need in improved synthetic protocols, which lead to the production of complex multiphosphorylated peptides. Specifically, there is a need of a process, which enables the synthesis of multiphosphorylated peptides with up to seven pSer and pThr residues, including such residues that are close in sequence.

### SUMMARY OF THE INVENTION

According to some embodiments, the present invention provides a method of Fmoc-solid phase synthesis of a peptide that comprises at least 3 phosphorylated amino acid (paa) residues, wherein at least one of the paa residues is a phosphorylated threonine (p-Thr) residue and wherein at least two of the paa residues are adjacent, the method comprising separately coupling at least 3 paas, wherein each paa coupling step comprises a coupling protocol, each coupling protocol comprises the parameters of: coupling duration, molar equivalents of paa and number of coupling cycles, wherein at least one of the paa coupling steps is microwave assisted at a temperature of 60° C. to 85° C., and wherein at least two coupling protocols differ in at least one of the parameters.

According to some embodiments, at least three couplings differ in at least one of the parameters.

According to some embodiments, the peptide consists of 10-25 amino acid residues.

According to other embodiments, the peptide consists of 12-18 amino acid residues.

According to some embodiments, the peptide comprises 3 to 7 paa residues.

According to some embodiments, the peptide comprises at least two p-Thr residues.

According to some embodiments, the peptide comprises at least two p-Thr residues and at least two phosphorylated Serine (p-Ser) residues.

According to some embodiments, each of the coupling steps is microwave assisted at a temperature of 60° C. to 85° C.

According to some embodiments, microwave assisted coupling is performed at about 75° C.

According to some embodiments, the peptide comprises at least two sub-sequences, wherein each sub-sequence comprises at least two adjacent paa residues, selected from p-Thr-p-Ser, p-Ser-p-Thr and p-Thr-p-Thr.

According to some embodiments, each coupling protocol comprises the parameters: coupling duration between 5 to 20 minutes, molar equivalents of paa between 3 and 6, and the number of coupling cycles between 1 to 3.

According to some embodiments, each coupling protocol comprises a step of Fmoc removal, comprising contacting the synthesized peptide with an amine at least once for a duration of 5-20 minutes at a temperature in the range of 10-80° C.

According to some embodiments, the first two phosphorylated residues in the peptide are coupled using 3 equivalents of phosphorylated amino acids for 5 minutes and one coupling cycle; the third and optionally forth phosphorylated amino acids in the peptide are couples using 3 equivalents of phosphorylated amino acids for 10 minutes and one coupling cycle; the fourth and/or fifth phosphorylated residues in the peptide are coupled using 3 equivalents of phosphorylated amino acids for 5 minutes and two coupling cycles; and the fifth and/or sixth and following phosphorylated residues in the peptide are coupled using 5 equivalents of phosphorylated amino acids for 5 minutes and two coupling cycles.

According to some embodiments, the at least two coupling protocols differ in at least one parameter, selected from: at least 50% difference in coupling duration, at least 3 minutes difference in coupling duration, at least 33% in molar equivalents of the paa, and the number of coupling cycles.

According to some embodiments, the method comprises at least a first paa coupling step and a second paa coupling step, wherein the first paa coupling step comprises coupling a first paa for a first duration, wherein the second paa coupling step comprises coupling a second paa for a second duration, and wherein the second duration is at least 50% higher than the first duration.

According to some embodiments, the method comprises at least a first paa coupling step and a second paa coupling step, wherein the first paa coupling step comprises coupling a first paa for a first duration, wherein the second paa coupling step comprises coupling a second paa for a second duration, and wherein the second duration is at least 3 minutes longer than the first duration.

According to some embodiments, the method comprises at least a first paa coupling step and a second paa coupling step, wherein the first paa coupling step comprises coupling a first paa using a first predetermined value of molar equivalents of the first paa, wherein the second paa coupling step comprises coupling a second paa using a second predetermined value of molar equivalents of the second paa, and wherein the second predetermined value is at least 33% larger than the second predetermined value.

According to some embodiments, the method comprises at least a first paa coupling step and a second paa coupling step, wherein the first paa coupling step comprises coupling a first paa a first number of coupling cycles, wherein the second paa coupling step comprises coupling a second paa a second number of coupling cycles, and wherein the second number of coupling cycles is larger than the first number of coupling cycles by at least 1.

According to some embodiments, the first paa coupling step comprises coupling the first paa once and the second paa coupling step comprises coupling the second paa twice.

According to some embodiments, the present invention provides a multi-phosphorylated peptide synthesized according to the method disclosed herein.

According to some embodiments, the peptide is selected from the group consisting of: SEQ ID NO. 1, SEQ ID NO. 2, SEQ ID NO. 3, SEQ ID NO. 5, SEQ ID NO. 6, SEQ ID NO. 8, SEQ ID NO. 9, SEQ ID NO. 10 and SEQ ID NO. 11.

### BRIEF DESCRIPTION OF THE FIGS

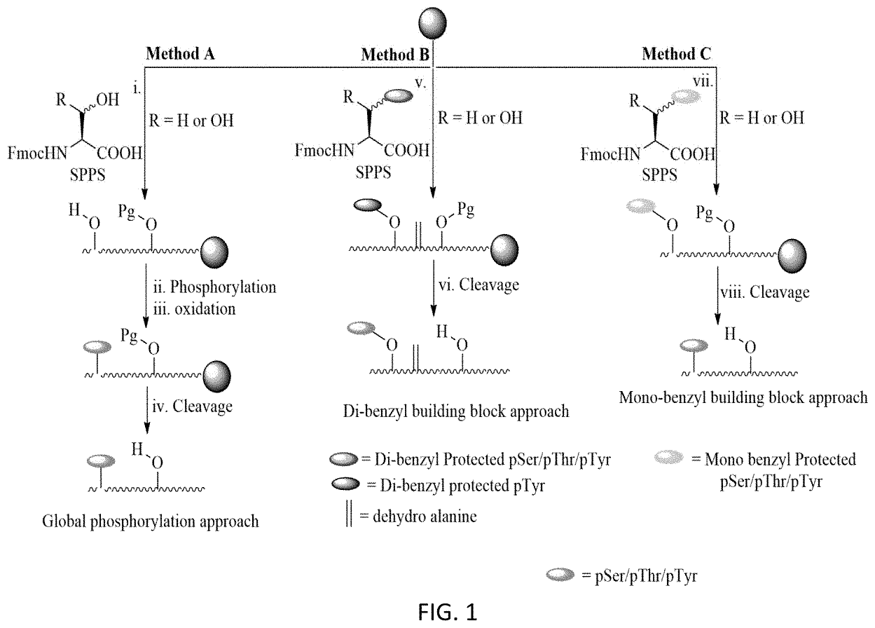

FIG. 1 illustrates schemes of methods for the synthesis of phosphorylated peptides: the global phosphorylation approach (Method A); and the building block approach (Methods B and C).

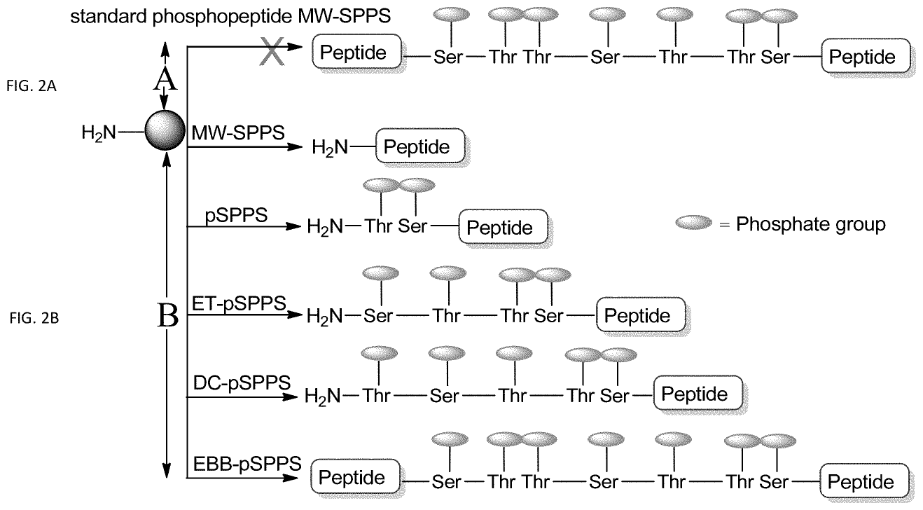

FIG. 2A illustrates synthetic protocols employing previously reported conditions.

FIG. 2B illustrates synthetic protocols according to some embodiments of the current invention.

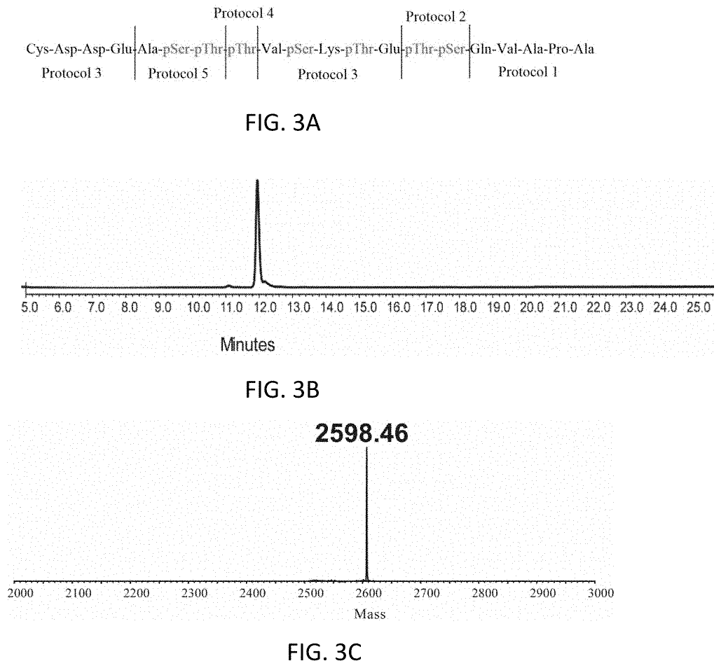

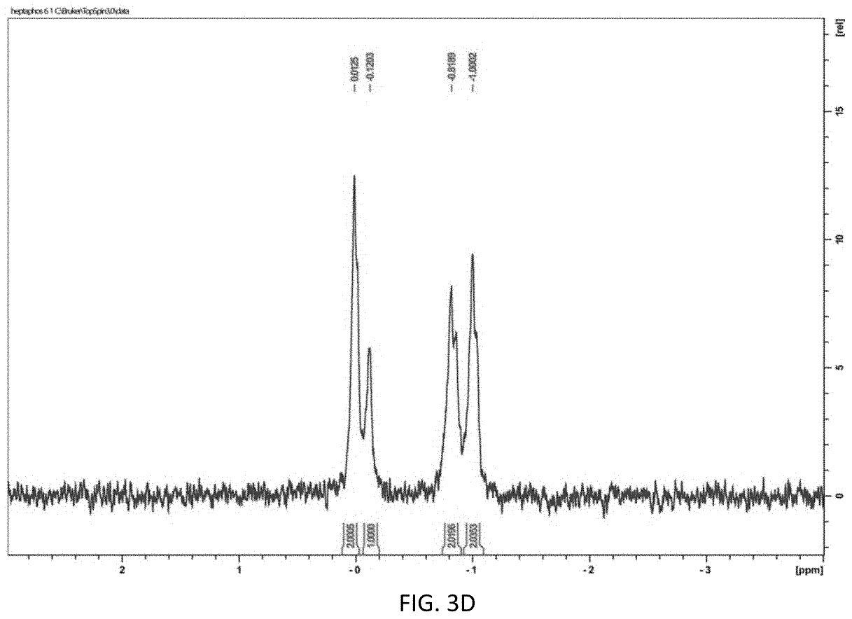

FIG. 3A illustrates the sequence of protocols used in the synthesis of Peptide 1 (SEQ ID NO. 1).

FIG. 3B illustrates a reverse phase ultra-performance liquid chromatography (RP-UPLC) chromatogram of Peptide 1 (SEQ ID NO. 1).

FIG. 3C illustrates a deconvoluted mass spectra of Peptide 1 (SEQ ID NO. 1).

FIG. 3D illustrates a ³¹P NMR spectra of Peptide 1 (SEQ ID NO. 1).

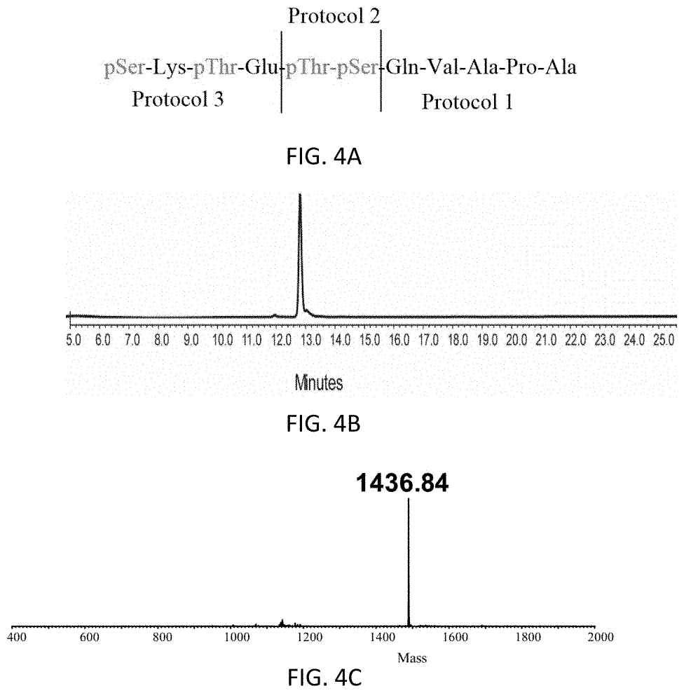

FIG. 4A illustrates the protocols used in the synthesis of Peptide 2 (SEQ ID NO. 2).

FIG. 4B illustrates an RP-UPLC chromatogram of Peptide 2 (SEQ ID NO. 2).

FIG. 4C illustrates a deconvoluted mass spectra of Peptide 2 (SEQ ID NO. 2).

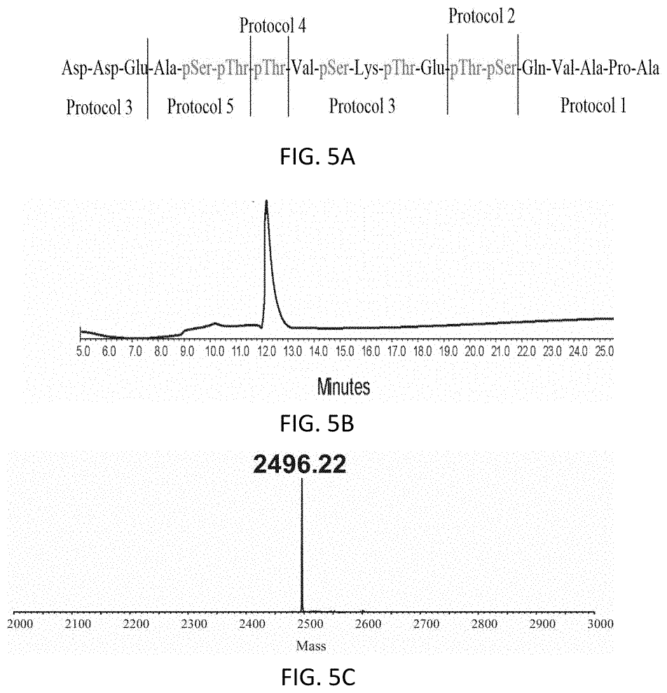

FIG. 5A illustrates the protocols used in the synthesis of Peptide 3 (SEQ ID NO. 3).

FIG. 5B illustrates an RP-UPLC chromatogram of Peptide 3 (SEQ ID NO. 3).

FIG. 5C illustrates a deconvoluted mass spectra of Peptide 3 (SEQ ID NO. 3).

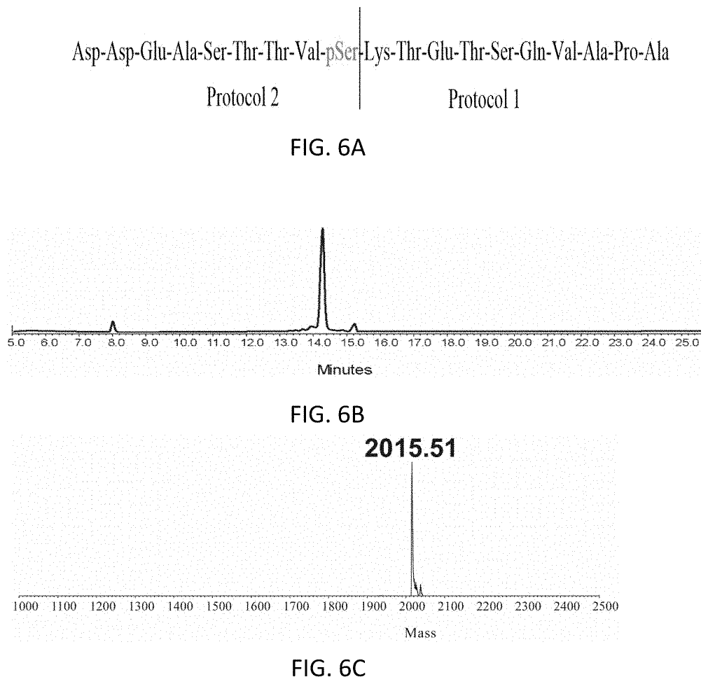

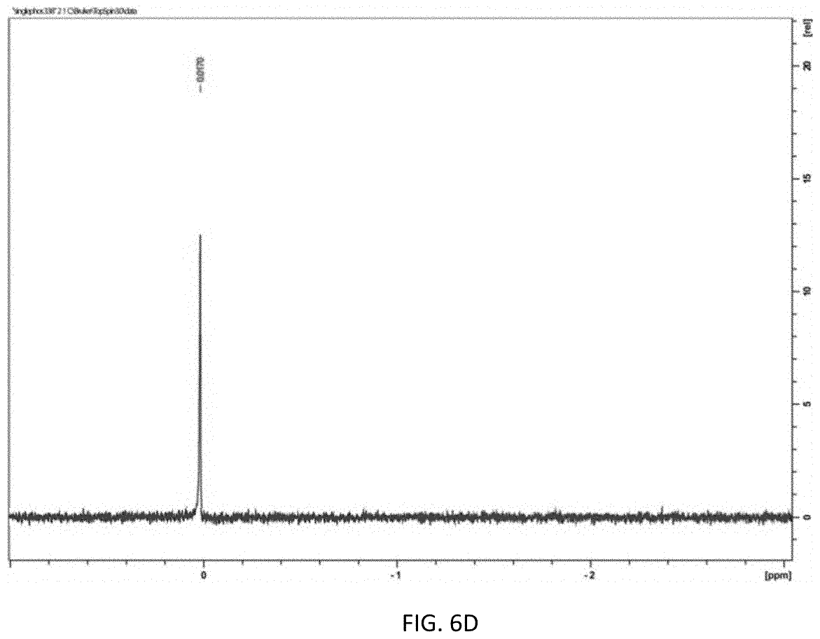

FIG. 6A illustrates the protocols used in the synthesis of Peptide 4 (SEQ ID NO. 4).

FIG. 6B illustrates an RP-UPLC chromatogram of Peptide 4 (SEQ ID NO. 4).

FIG. 6C illustrates a deconvoluted mass spectra of Peptide 4 (SEQ ID NO. 4).

FIG. 6D illustrates a ³¹P NMR spectra of Peptide 4 (SEQ ID NO. 4)

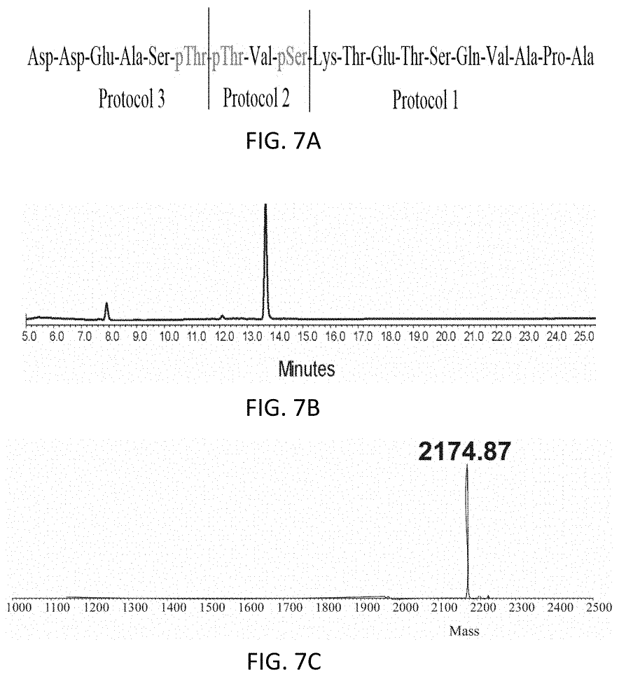

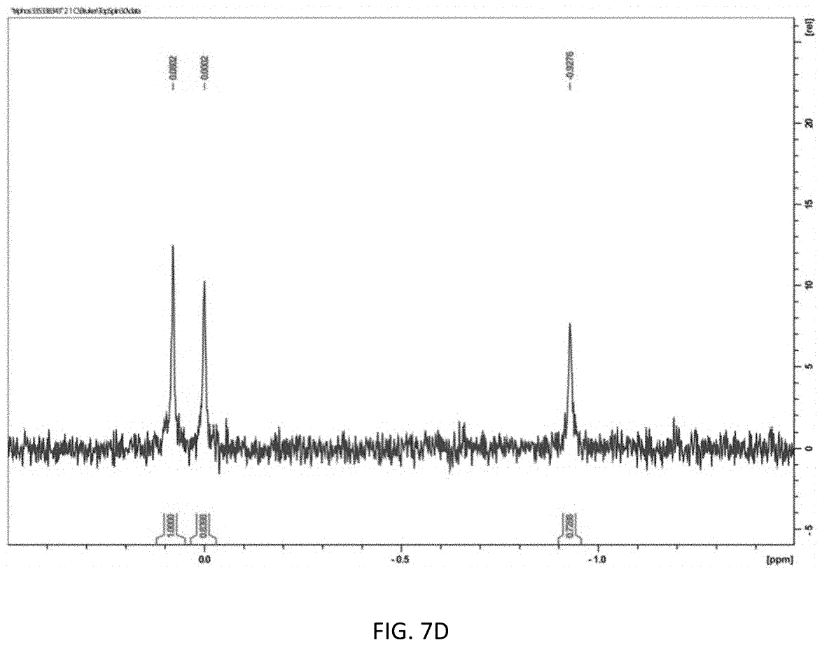

FIG. 7A illustrates the different protocols used in the synthesis of Peptide 5 (SEQ ID NO. 5).

FIG. 7B illustrates an RP-UPLC chromatogram of Peptide 5 (SEQ ID NO. 5).

FIG. 7C illustrates a deconvoluted mass spectra of Peptide 5 (SEQ ID NO. 5).

FIG. 7D illustrates a ³¹P NMR spectra of Peptide 5 (SEQ ID NO. 5).

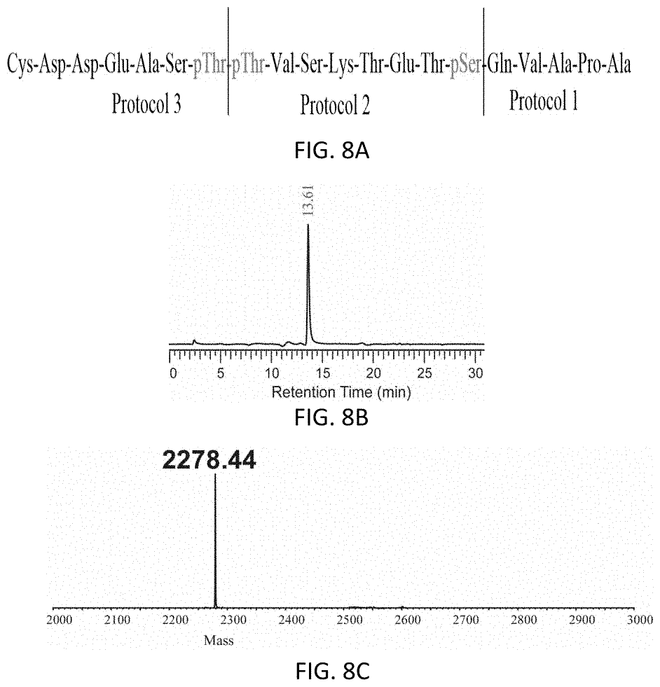

FIG. 8A illustrates the different protocols used in the synthesis of Peptide 6 (SEQ ID NO. 6).

FIG. 8B illustrates an RP-UPLC chromatogram of Peptide 6 (SEQ ID NO. 6).

FIG. 8C illustrates a deconvoluted mass spectra of Peptide 6 (SEQ ID NO. 6).

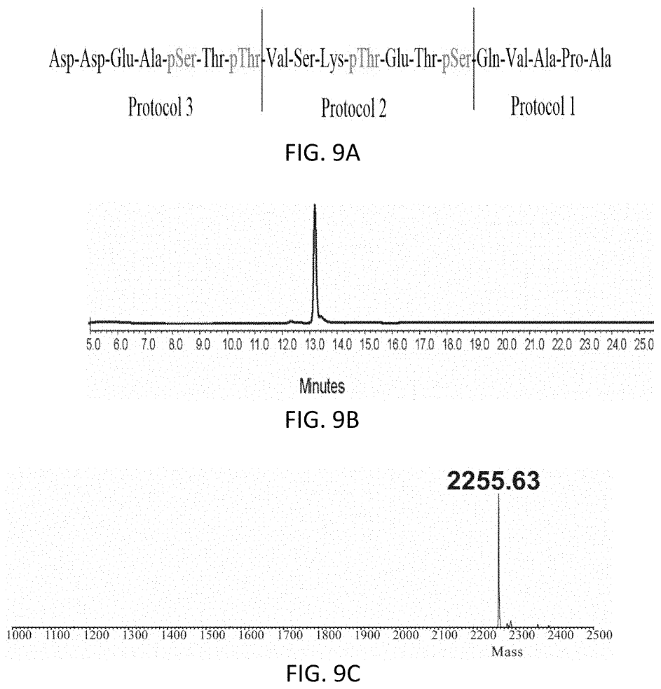

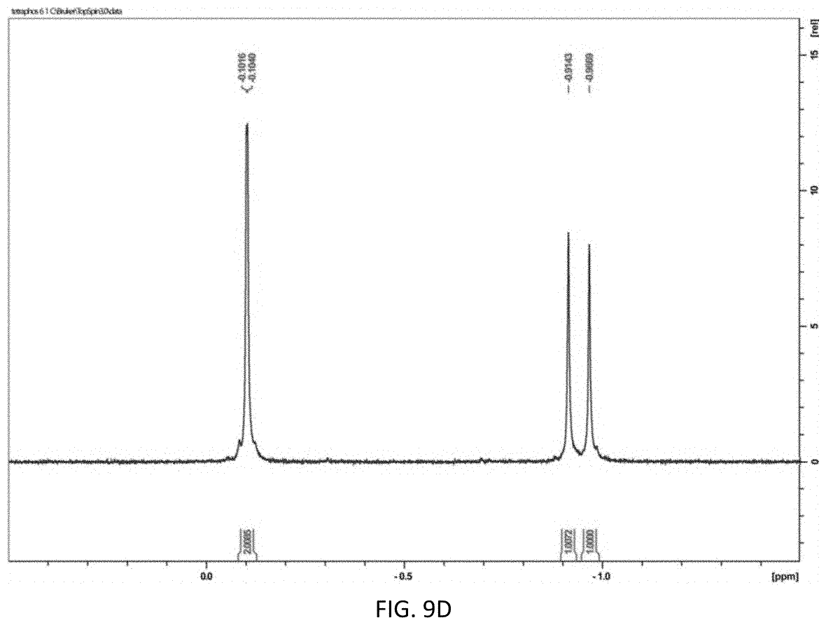

FIG. 9A illustrates the different protocols used in the synthesis of Peptide 7 (SEQ ID NO. 7).

FIG. 9B illustrates an RP-UPLC chromatogram of Peptide 7 (SEQ ID NO. 7).

FIG. 9C illustrates a deconvoluted mass spectra of Peptide 7 (SEQ ID NO. 7).

FIG. 9D illustrates a ³¹P NMR spectra of Peptide 7 (SEQ ID NO. 7).

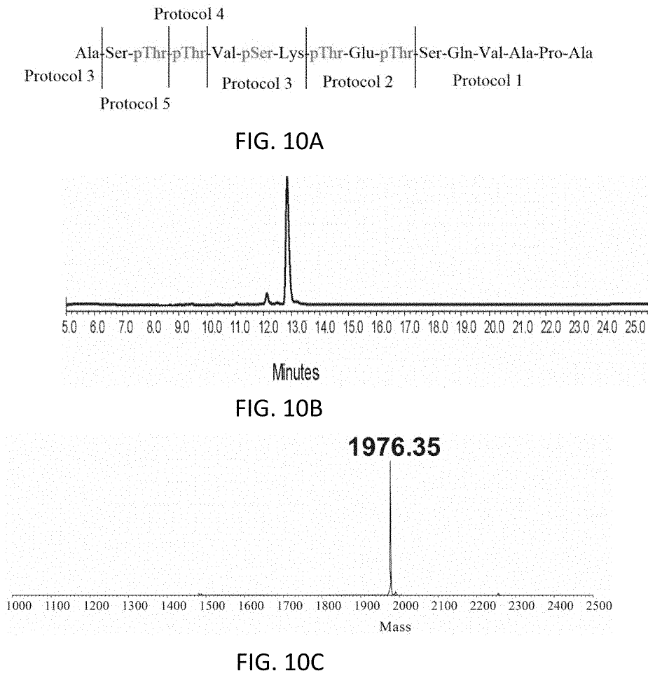

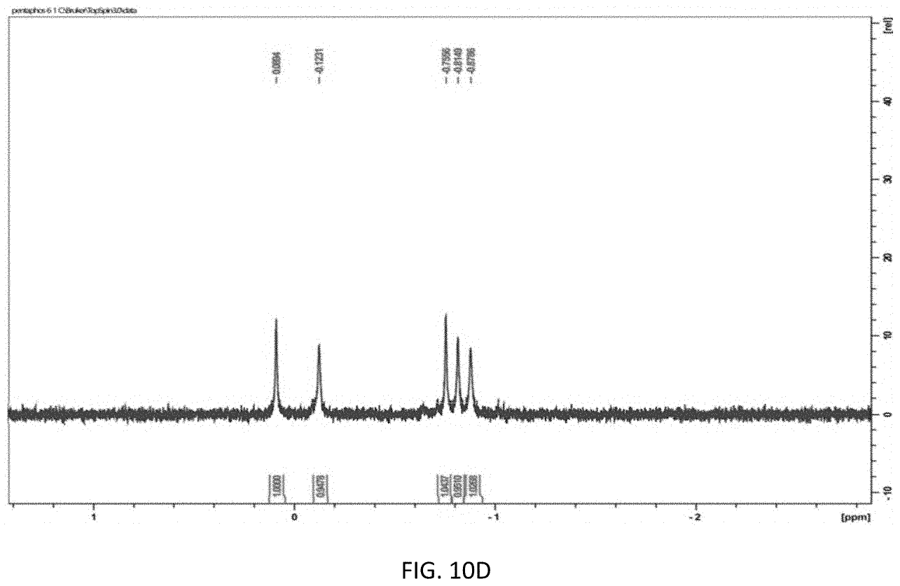

FIG. 10A illustrates the different protocols used in the synthesis of Peptide 8 (SEQ ID NO. 8).

FIG. 10B illustrates an RP-UPLC chromatogram of Peptide 8 (SEQ ID NO. 8).

FIG. 10C illustrates a deconvoluted mass spectra of Peptide 8 (SEQ ID NO. 8).

FIG. 10D illustrates a ³¹P NMR spectra of Peptide 8 (SEQ ID NO. 8).

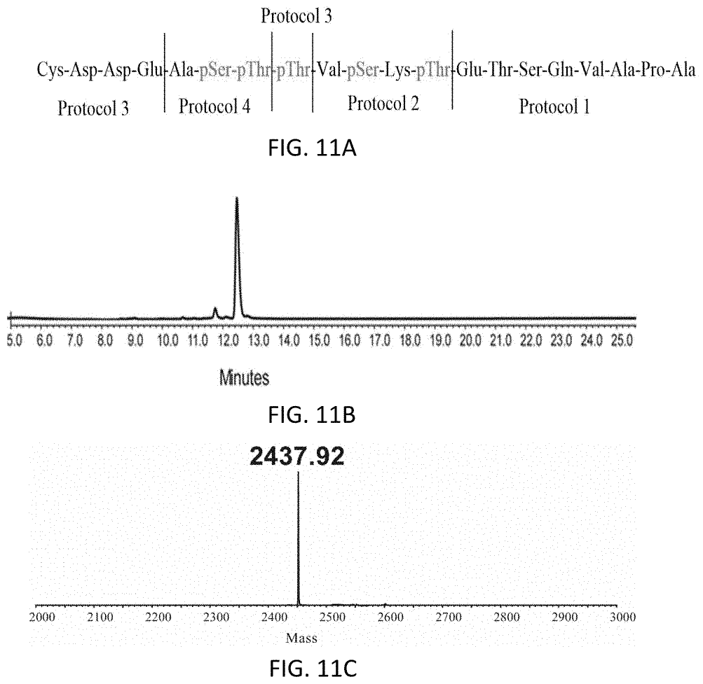

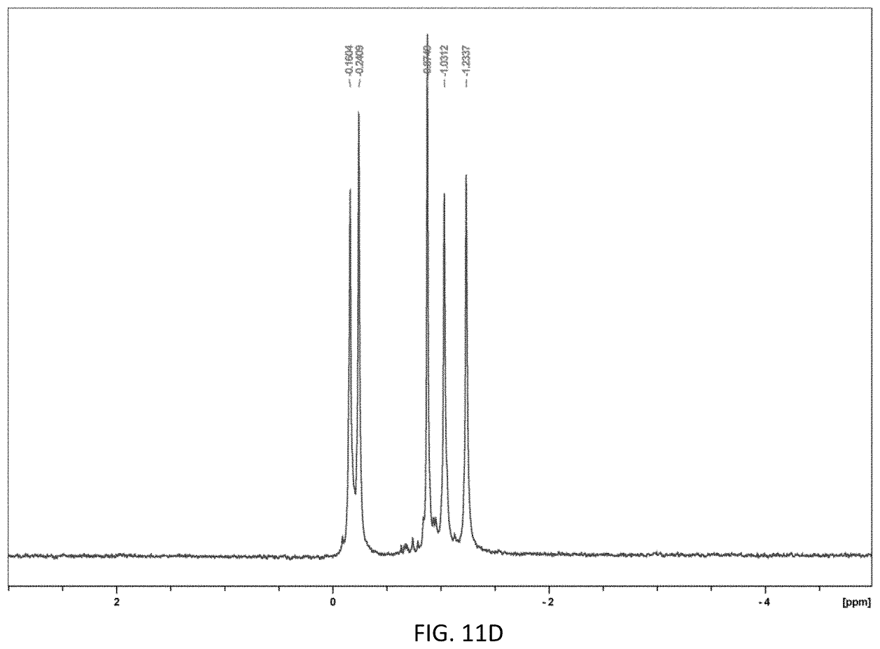

FIG. 11A illustrates the different protocols used in the synthesis of Peptide 9 (SEQ ID NO. 9).

FIG. 11B illustrates an RP-UPLC chromatogram of Peptide 9 (SEQ ID NO. 9).

FIG. 11C illustrates a deconvoluted mass spectra of Peptide 9 (SEQ ID NO. 9).

FIG. 11D illustrates a ³¹P NMR spectra of Peptide 9 (SEQ ID NO. 9)

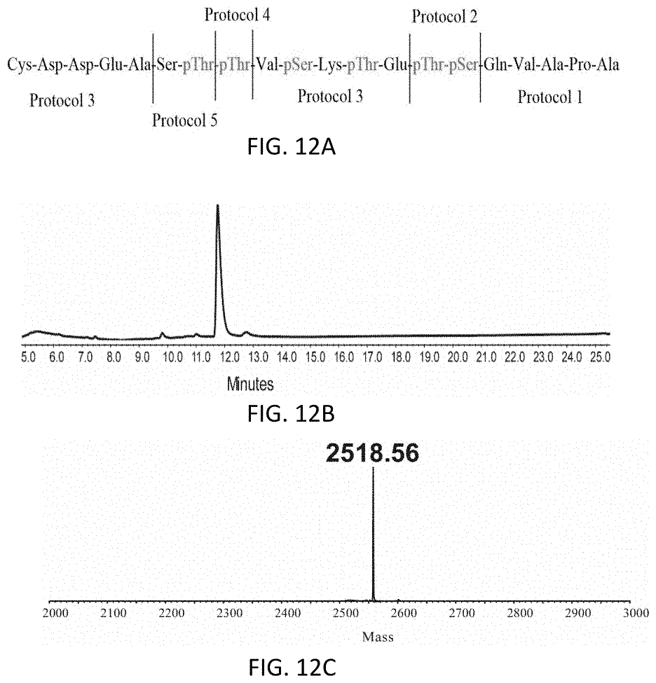

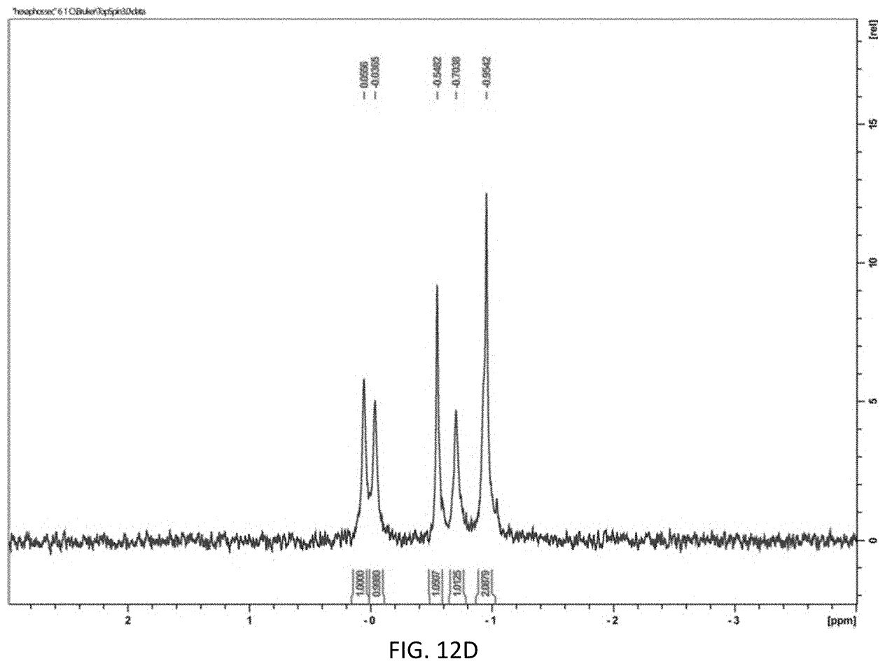

FIG. 12A illustrates the protocols used in the synthesis of Peptide 10 (SEQ ID NO. 10).

FIG. 12B illustrates an RP-UPLC chromatogram of Peptide 10 (SEQ ID NO. 10).

FIG. 12C illustrates a deconvoluted mass spectra of Peptide 10 (SEQ ID NO. 10).

FIG. 12D illustrates a ³¹P NMR spectra of Peptide 10 (SEQ ID NO. 10)

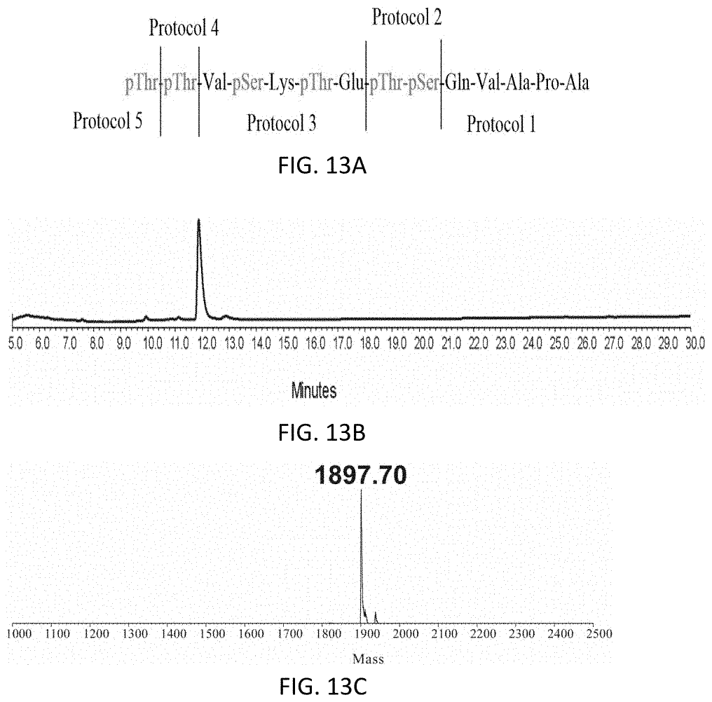

FIG. 13A illustrates the protocols used in the synthesis of Peptide 11 (SEQ ID NO. 11).

FIG. 13B illustrates an RP-UPLC chromatogram of Peptide 11 (SEQ ID NO. 11).

FIG. 13C illustrates a deconvoluted mass spectra of Peptide 11 (SEQ ID NO. 11).

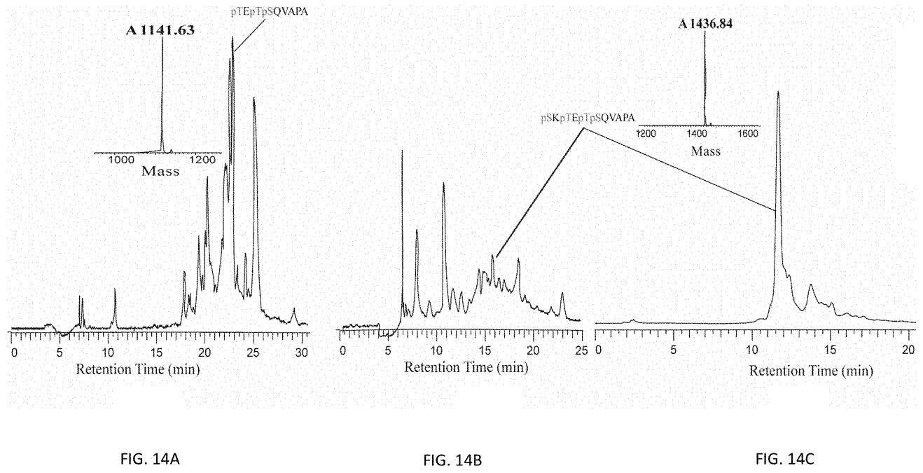

FIG. 14A illustrates an RP-HPLC chromatogram of the peptide SEQ ID NO. 12 synthesized employing previously reported conditions.

FIG. 14B illustrates an RP-HPLC chromatogram of the tetra phosphorylated peptide SEQ ID NO. 2 synthesized using previously reported conditions.

FIG. 14C illustrates an RP-HPLC chromatogram of the tetra phosphorylated peptide SEQ ID NO. 2 synthesized employing the method of the current invention.

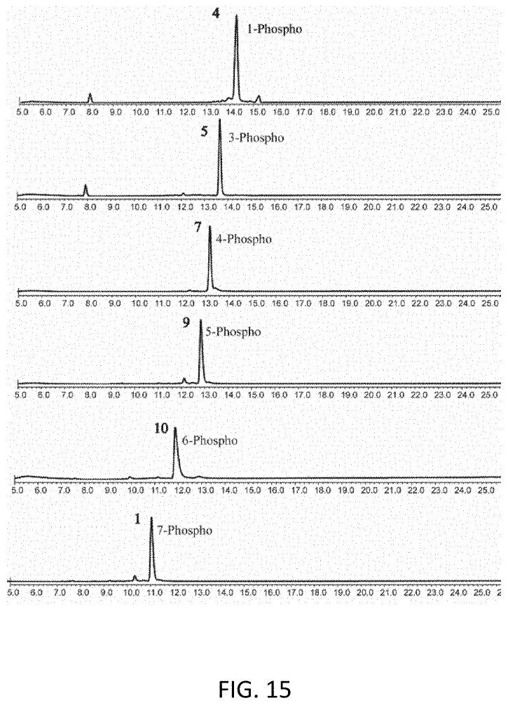

FIG. 15 illustrates an overlay of UPLC profiles of the peptides with increasing number of phosphorylation sites.

### DETAILED DESCRIPTION OF THE INVENTION

According to some embodiments, the current invention provides a synthetic method for the preparation of phosphorylated amino acid sequences. Specifically, there is provided a method of Fmoc-solid phase peptide synthesis (SPPS), which is highly efficient and economical in the preparation of various phosphorylated amino acid (paa) sequences, including complicated sequences that comprise at least 3 phosphorylated amino acid residues, wherein at least one of the paa residues is a phosphorylated threonine (p-Thr) residue and wherein at least two of the paa residues are adjacent. The method applies different coupling protocols for each amino acid in the sequence by adjusting the coupling conditions as required. according to some embodiments, the method comprises separately coupling at least 3 paas, wherein each coupling step comprises a coupling protocol. According to some embodiments, each coupling protocol comprises the parameters of: coupling duration, molar equivalents of paa and number of coupling cycles. According to some embodiments, at least one of the coupling steps is microwave assisted at a temperature of 60° C. to 85° C. According to some embodiments, at least two coupling protocols differ in at least one of the parameters. Preferably, at least three coupling protocols differ in at least one of the parameters.

The method of the current invention was used to synthesize a series of Rhodopsin derived peptides with up to seven phosphorylated Ser and Thr residues. The peptides were obtained in high yield and purity. According to some embodiments, each coupling protocol is designed based on different parameters, such as the specific phosphorylation pattern and the location of the added paa in the sequence. The results show that adjusting the coupling conditions based on the specific phosphorylation pattern is the key for the successful synthesis of multiphosphorylated peptides that could not be synthesized using any other protocol. The current method paves the way for the efficient and routine synthesis of multi-phosphorylated peptides of biological interest.

According to some embodiments, there is provided a method of Fmoc-solid phase peptide synthesis of a sequence that comprises at least 3 phosphorylated amino acid (paa) residues, wherein at least one of the paa residues is a phosphorylated threonine (p-Thr) residue and wherein at least two of the paa residues are adjacent, the method comprising separately coupling at least 3 paas, wherein each coupling step comprises a coupling protocol, each coupling protocol comprises the parameters of: coupling duration, molar equivalents of paa and number of coupling cycles, wherein at least one of the coupling steps is microwave assisted at a temperature of 60° C. to 85° C., and wherein at least two coupling protocols differ in at least one of the parameters.

It is to be understood that the phrase “wherein at least two coupling protocols differ in at least one of the parameters” refers to two or more protocols, which are different one from the other. The difference may be in one of the parameters or in two of the parameters. For example, the protocol termed in the Example section ‘pSPPS’ involves microwave one cycle of coupling of 3 molar equivalents the paa to be coupled at 75° C. for 5 minutes; and the protocol termed in the Example section ‘Et-pSPPS’ involves one cycle of microwave coupling of 3 molar equivalents the paa to be coupled at 75° C. for 10 minutes. The protocols pSPPS and Et-pSPPS as considered different under the terminology of the current disclosure, although the parameters of temperature, heating method (i.e. microwave), molar equivalent ratio. Specifically, these protocols are different in the one parameter, which is the coupling reaction duration. In another example, the protocol termed in the Example section ‘DC-pSPPS’ involves two cycles of microwave coupling of 3 molar equivalents the paa to be coupled at 75° C. for 5 minutes. The protocol DC-pSPPS differs from the pSPPS protocol by the number of coupling cycles (two in DC-pSPPS, compared to one cycle in pSPPS), and differs from the Et-pSPPS protocol by the number of coupling cycles (two in DC-pSPPS, compared to one cycle in Et pSPPS) and the coupling reaction duration (5 minutes in DC-pSPPS, compared to 10 minutes in Et pSPPS). Thus, the protocol DC-pSPPS is different from each of the pSPPS and the Et pSPPS protocols. In addition, in the example, where a solid phase peptide synthesis method comprises each one of the pSPPS, Et pSPPS and DC-pSPPS protocols, it is said that couplings differ in at least one of the parameters.

According to some embodiments, the method comprises at least three couplings which differ in at least one of the parameters. According to some embodiments, at least three couplings differ in at least one of the parameters. According to some embodiments, three couplings differ in at least one of the parameters. According to some embodiments, at least four couplings differ in at least one of the parameters. According to some embodiments, four couplings differ in at least one of the parameters.

It is to be understood that the terms “differ” and “difference” as used herein refer to a difference of at least 10%, or preferably at least 20%. For example, if a first coupling is performed for a coupling duration of 10 minutes, a second coupling is considered different if it is performed for at least 11 minutes or not more than 9 minutes. For another example, if a first coupling is performed using 5 equivalents of the paa, a second coupling is considered different if it is performed using at least 5.5 equivalents of the paa or not more than 4.5 equivalents of the paa. For another example, a coupling which is performed for one cycle is considered different than coupling which is performed for two cycles, as the difference between the coupling cycles in each protocol is 100%. It should be also understood that “a first coupling” does not necessary refer to the first, or C-terminal amino acid residue in the sequence, but to a first coupling reaction according to the methods of the present invention. The first coupling maybe therefore performed for coupling a phosphorylated amino acid to a solid-phase-bound amino acid or peptide.

Any solid support, known in art for SPPS, may be used in the synthetic methods of the present invention. This includes but non-limited to Rink amide MBHA polystyrene resin.

The method of the current invention, combining different MW-assisted coupling protocols for synthesis of multiphosphorylated peptides, is substantially different compared to known methods, which use a repeating protocol throughout the synthesis. The two main known method are the global phosphorylation and the building block approach. FIG. 1 depicts typical conditions routinely used for employing these methods.

The global phosphorylation approach proceeds in two steps: (i) Insertion of unprotected Ser/Thr/Tyr residues during SPPS; (ii) Post SPPS phosphorylation using di-tertbutyl or dibenzyl protected N,N-dialkyl/aryl phosphoramidites followed by oxidation with meta-chloro peroxybenzoic acid(m-CPBA)/tBuOOH/I₂ (FIG. 1, Method A, reference 23). The global approach has several drawbacks that limit its use: it cannot be automated, and is incompatible with the oxidation prone amino acids Met and Cys (24-25).

The building block approach, which is the commonly used synthetic strategy, utilizes Fmoc protected phosphorylated tyrosine, threonine or serine that are introduced at the required position during SPPS (FIG. 1, Methods B and C). Several protecting groups were studied for the synthesis of phosphorylated Tyr, Thr and Ser (26). The di-benzyl protected Fmoc-Tyr(PO₃Bzl₂)-OH amino acids are used for the synthesis of peptides with phosphorylated tyrosine (pTyr) in Fmoc-SPPS (FIG. 1, Method B, references 27-29).

Synthesis of peptides containing Thr and pSer using the di-benzyl phosphate protection is inefficient due to the significant β-elimination that takes place during Fmoc deprotection steps (FIG. 1, Method B, references 30-32). The introduction of the mono-benzyl protected amino acids, Fmoc-Ser(HPO₃Bzl)-OH and Fmoc-Thr(HPO₃Bzl)-OH, significantly improved the synthesis of peptides with pSer and pThr as it minimized the formation of the β-elimination side products (FIG. 1, Method C, references 14, 33, 34). Thus, these monobenzyl protected pThr and pSer amino acids are now routinely used for the SPS of phosphorylated peptides.

The Current invention describes, according to some embodiments, a general and efficient method for synthesizing multi phosphorylated peptides, containing several pSer and pThr residues that are close in the sequence, by applying multiple coupling protocols instead of a single coupling protocol as previously described. The strategy involves multiple microwave (MW) assisted coupling protocols, which were designed for based on the unique phosphorylation pattern of each peptide. It is shown that this strategy enables the efficient synthesis of a series Rho330-348 multi phosphorylated peptide library with different combinations of phosphorylation patterns, making the current method a general one for the multi phosphorylated peptide synthesis.

The synthesis of peptides bearing multiple and adjacent pThr residues, such as Rho 330-340, is even more difficult than the synthesis of peptides with well separated paas or the ones containing only pSer amino acids. MW-assisted Fmoc-SPPS of such peptides was never reported. Most reports describing the MW-assisted SPPS of multi-phosphorylated peptides use identical coupling conditions for the entire synthesis (FIG. 2A). I was found that such strategy is highly inefficient for peptides bearing multiple pThr moieties or when the pAAs are in close proximity (FIG. 2A). Extended coupling times and a large excess of expensive reagents may be used for the synthesis of one multi-phosphorylated peptide in a very small quantity, but this is not a viable approach for the synthesis of a library of multi-phosphorylated peptides.

To address the above problems and demonstrate a new solution to the need in multiphosphopeptides with numerous phosphorylations in close proximity, a novel strategy is disclosed herein for the MW-assisted Fmoc-SPPS of a multi-phosphorylated peptide library and applied it for the synthesis of a library of Rho 330-340 derived peptides. The method of the current invention employs the combination of different coupling methods that differ in their coupling duration, the equivalents of pAAs and the number of repeating coupling cycles, according to some embodiments, (FIG. 2B).

The method was employed for synthesizing the most heavily phosphorylated Rho 330-340 peptide (SEQ ID NO. 1), which contains seven pAAs. These conditions were then applied for the other multiphosphopeptides in a library.

Thus, the present invention concerns a method for synthesizing multiphosphorylated peptides the method comprising: applying to the peptide multiple (at least two) different coupling protocols. According to some embodiments, the current invention provides a method of Fmoc-solid phase synthesis of a peptide that comprises at least 3 phosphorylated amino acid (paa) residues, wherein at least one of the paa residues is a phosphorylated threonine (p-Thr) residue and wherein at least two of the paa residues are adjacent, the method comprising separately coupling at least 3 paas, wherein each coupling step comprises a coupling protocol, each coupling protocol comprises the parameters of: coupling duration, molar equivalents of paa and number of coupling cycles, wherein at least one of the coupling step is microwave assisted at a temperature of 60° C. to 85° C., and wherein at least two coupling protocols differ in at least one of the parameters.

According to some embodiments, at least three couplings differ in at least one of the parameters. Preferably the peptide is a multiphosphorylated peptide having more than three phosphorylated amino acid residues, preferably more than 4, 5 6 or 7 phosphorylated amino acid residues.

According to some embodiments, the peptide sequence comprises at least 4 phosphorylated amino acid (paa) residues. According to some embodiments, the peptide sequence comprises 4 phosphorylated amino acid residues. According to some embodiments, the peptide sequence comprises at least 5 phosphorylated amino acid (paa) residues. According to some embodiments, the peptide sequence comprises 5 phosphorylated amino acid residues. According to some embodiments, the peptide sequence comprises at least 6 phosphorylated amino acid (paa) residues. According to some embodiments, the peptide sequence comprises 6 phosphorylated amino acid residues. According to some embodiments, the peptide sequence comprises at least 7 phosphorylated amino acid (paa) residues. According to some embodiments, the peptide sequence comprises 7 phosphorylated amino acid residues.

According to some embodiments, the peptide comprises 3 to 7 paa residues. According to some embodiments, the peptide comprises 4 to 7 paa residues. According to some embodiments, the peptide comprises 5 to 7 paa residues. According to some embodiments, the peptide comprises 6 to 7 paa residues.

According to some embodiments, the peptide comprises at least two p-Thr residues. According to some embodiments, the peptide comprises at least two phosphorylated Serine (p-Ser) residues. According to some embodiments, the peptide comprises at least two p-Thr residues and at least two p-Ser residues. According to some embodiments, the peptide comprises at least three p-Thr residues. According to some embodiments, the peptide comprises at least four p-Thr residues.

According to some embodiments, the peptide comprises at least one sub-sequence comprising at least two adjacent paa residues, selected from p-Thr-p-Ser, p-Ser-p-Thr and p-Thr-p-Thr. According to some embodiments, the peptide comprises at least one sub-sequence comprising adjacent p-Thr-p-Ser residue. According to some embodiments, the peptide comprises at least one sub-sequence comprising adjacent p-Ser-p-Thr residue. According to some embodiments, the peptide comprises at least one sub-sequence comprising adjacent p-Thr-p-Thr residue.

According to some embodiments, the peptide comprises at least two sub-sequences, wherein each sub-sequence comprises at least two adjacent paa residues, selected from p-Thr-p-Ser, p-Ser-p-Thr and p-Thr-p-Thr.

It is to be understood that sub-sequences, which comprises three adjacent paa residues is considered to include two sub-sequences comprising at least two adjacent paa residues—the sub-sequence of the first and second paa residue, and the sub-sequence of the second and third paa residue. For example, a peptide, which includes the sub-sequence p-Ser-p-Thr-p-Thr is considered to include the adjacent p-Ser-p-Thr residue and the adjacent p-Thr-p-Thr residue. Similarly, a peptide, which includes the sub-sequence p-Thr-p-Thr-p-Ser is considered to include the adjacent p-Thr-p-Ser residue and the adjacent p-Thr-p-Thr residue.

According to some embodiments, the peptide comprises at least one sub-sequence comprising three adjacent paa residues. According to some embodiments, the three adjacent paa residues include at least one p-Thr residue. According to some embodiments, the three adjacent paa residues include at least one p-Ser residue. According to some embodiments, the three adjacent paa residues include at least one p-Ser residue and at least one p-Thr residue. According to some embodiments, the three adjacent paa residues include one p-Ser residue and two p-Thr residues. According to some embodiments, peptide consists of 5-50 amino acid residues. According to some embodiments, peptide consists of 7-35 amino acid residues. According to some embodiments, peptide consists of 10-25 amino acid residues. According to some embodiments, peptide consists of 15-25 amino acid residues.

It is to be understood that phosphorylated amino acid residues are counted in the total count of amino acid residues in the peptide. For example, a peptide consisting of 12 unmodified amino acid residues and 5 paa residues is considered to consist of 17 amino acid residues, according to some embodiments.

According to some embodiments, the coupling protocols are microwave assisted Fmoc-SPPS synthetic protocols.

According to some embodiments, at least one of the paa coupling steps is microwave assisted at a temperature of 60° C. to 85° C. According to some embodiments, at least two of the paa coupling steps is microwave assisted at a temperature of 60° C. to 85° C. According to some embodiments, at least three of the paa coupling steps is microwave assisted at a temperature of 60° C. to 85° C. According to some embodiments, at least four of the paa coupling steps is microwave assisted at a temperature of 60° C. to 85° C. According to some embodiments, each of the paa coupling steps is microwave assisted at a temperature of 60° C. to 85° C.

According to some embodiments, at least one of the paa coupling steps is microwave assisted at a temperature of 70° C. to 80° C. According to some embodiments, at least two of the paa coupling steps is microwave assisted at a temperature of 70° C. to 80° C. According to some embodiments, at least three of the paa coupling steps is microwave assisted at a temperature of 70° C. to 80° C. According to some embodiments, at least four of the paa coupling steps is microwave assisted at a temperature of 70° C. to 80° C. According to some embodiments, each of the paa coupling steps is microwave assisted at a temperature of 70° C. to 80° C.

According to some embodiments, at least one of the paa coupling steps is microwave assisted at a temperature of 75° C. According to some embodiments, at least two of the paa coupling steps is microwave assisted at a temperature of 75° C. According to some embodiments, at least three of the paa coupling steps is microwave assisted at a temperature of 75° C. According to some embodiments, at least four of the paa coupling steps is microwave assisted at a temperature of 75° C. According to some embodiments, each of the paa coupling steps is microwave assisted at a temperature of 75° C.

According to some embodiments, of the coupling protocols for the phosphorylated amino used, the at least two protocols differ from each other by at least one of the following parameters: (1) coupling time or duration (2) number of coupling cycles, (3) number of equivalents of phosphorylated amino acid used.

Nevertheless, the different protocols may share common ranges of each of the parameters or some of the parameters, according to some embodiments. According to some embodiments, at least two paa coupling protocols comprise the parameter of coupling duration between 5 to 20 minutes. According to some embodiments, at least two paa coupling protocols comprise the parameter of coupling duration between 5 to 15 minutes. According to some embodiments, at least two paa coupling protocols comprise the parameter of coupling duration between 5 to 10 minutes. According to some embodiments, at least three paa coupling protocols comprise the parameter of coupling duration between 5 to 20 minutes. According to some embodiments, at least three paa coupling protocols comprise the parameter of coupling duration between 5 to 15 minutes. According to some embodiments, at least three paa coupling protocols comprise the parameter of coupling duration between 5 to 10 minutes. According to some embodiments, at least four paa coupling protocols comprise the parameter of coupling duration between 5 to 20 minutes. According to some embodiments, at least four paa coupling protocols comprise the parameter of coupling duration between 5 to 15 minutes. According to some embodiments, at least four paa coupling protocols comprise the parameter of coupling duration between 5 to 10 minutes. According to some embodiments, each of the paa coupling protocols comprise the parameter of coupling duration between 5 to 20 minutes. According to some embodiments, each of the paa coupling protocols comprise the parameter of coupling duration between 5 to 15 minutes. According to some embodiments, each of the paa coupling protocols comprise the parameter of coupling duration between 5 to 10 minutes.

According to some embodiments, at least two paa coupling protocols comprise the parameter of 2 to 10 paa equivalents. According to some embodiments, at least two paa coupling protocols comprise the parameter of 3 to 6 paa equivalents. According to some embodiments, at least two paa coupling protocols comprise the parameter of 3 to 5 paa equivalents. According to some embodiments, at least three paa coupling protocols comprise the parameter of 2 to 10 paa equivalents. According to some embodiments, at least three paa coupling protocols comprise the parameter of 3 to 6 paa equivalents. According to some embodiments, at least three paa coupling protocols comprise the parameter of 3 to 5 paa equivalents. According to some embodiments, at least four paa coupling protocols comprise the parameter of 2 to 10 paa equivalents. According to some embodiments, at least four paa coupling protocols comprise the parameter of 3 to 6 paa equivalents. According to some embodiments, at least four paa coupling protocols comprise the parameter of 3 to 5 paa equivalents. According to some embodiments, each of the paa coupling protocols comprise the parameter of 2 to 10 paa equivalents. According to some embodiments, each of the paa coupling protocols comprise the parameter of 3 to 6 paa equivalents. According to some embodiments, each of the paa coupling protocols comprise the parameter of 3 to 5 paa equivalents.

According to some embodiments, at least two paa coupling protocols comprise the parameter of 1 to 5 coupling cycles. According to some embodiments, at least two paa coupling protocols comprise the parameter of 1 to 3 coupling cycles. According to some embodiments, at least two paa coupling protocols comprise the parameter of 1 to 2 coupling cycles. According to some embodiments, at least 3 paa coupling protocols comprise the parameter of 1 to 5 coupling cycles. According to some embodiments, at least 3 paa coupling protocols comprise the parameter of 1 to 3 coupling cycles. According to some embodiments, at least 3 paa coupling protocols comprise the parameter of 1 to 2 coupling cycles. According to some embodiments, at least 4 paa coupling protocols comprise the parameter of 1 to 5 coupling cycles. According to some embodiments, at least 4 paa coupling protocols comprise the parameter of 1 to 3 coupling cycles. According to some embodiments, at least 4 paa coupling protocols comprise the parameter of 1 to 2 coupling cycles. According to some embodiments, each of the paa coupling protocols comprise the parameter of 1 to 5 coupling cycles. According to some embodiments, each of the paa coupling protocols comprise the parameter of 1 to 3 coupling cycles. According to some embodiments, each of the paa coupling protocols comprise the parameter of 1 to 2 coupling cycles.

According to some embodiments, at least two paa coupling protocols comprise the parameters of coupling duration between 5 to 20 minutes, molar equivalents of paa between 3 and 6, and the number of coupling cycles between 1 to 3. According to some embodiments, at least 3 paa coupling protocols comprise the parameters of coupling duration between 5 to 20 minutes, molar equivalents of paa between 3 and 6, and the number of coupling cycles between 1 to 3. According to some embodiments, at least 4 paa coupling protocols comprise the parameters of coupling duration between 5 to 20 minutes, molar equivalents of paa between 3 and 6, and the number of coupling cycles between 1 to 3. According to some embodiments, each of the paa coupling protocols comprise the parameters of coupling duration between 5 to 20 minutes, molar equivalents of paa between 3 and 6, and the number of coupling cycles between 1 to 3.

According to some embodiments, at least two paa coupling protocols comprise the parameters of coupling duration between 5 to 10 minutes, molar equivalents of paa between 3 and 5, and the number of coupling cycles between 1 to 2. According to some embodiments, at least 3 paa coupling protocols comprise the parameters of coupling duration between 5 to 10 minutes, molar equivalents of paa between 3 and 5, and the number of coupling cycles between 1 to 2. According to some embodiments, at least 4 paa coupling protocols comprise the parameters of coupling duration between 5 to 10 minutes, molar equivalents of paa between 3 and 5, and the number of coupling cycles between 1 to 2. According to some embodiments, each of the paa paa coupling protocols comprise the parameters of coupling duration between 5 to 10 minutes, molar equivalents of paa between 3 and 5, and the number of coupling cycles between 1 to 2.

Preferably the method is for the preparation of a peptide of at least 3 or at least 4 phosphorylated residues using three different coupling protocols. More preferably the peptide has 5 phosphorylated residues and four different protocols are used. More preferably the peptide has 6 or more phosphorylated residues and in that case five different protocols are used.

None limiting guidelines for using the protocols are given below:

For the first two phosphorylated amino acids in the peptide it is preferable to use the protocol pSPPS, according to some embodiments. Specifically, the protocol pSPPS involves one cycle of MW coupling at 75° C. for 5 minutes using 3 equivalents of paa. According to some embodiments, each of the coupling protocols for the steps of coupling the first two paas in the peptide comprises the parameter of coupling duration in the range of 2.5 to 7.5 minutes. According to some embodiments, each of the coupling protocols for the steps of coupling the first two paas in the peptide comprises the parameter of coupling duration of about 5 minutes. According to some embodiments, each of the coupling protocols for the steps of coupling the first two paas in the peptide comprises the parameter of 2-4 equivalents of paa. According to some embodiments, each of the coupling protocols for the steps of coupling the first two paas in the peptide comprises the parameter of about 3 equivalents of paa. According to some embodiments, each of the coupling protocols for the steps of coupling the first two paas in the peptide comprises the parameter of one coupling cycle.

For the third phosphorylated amino acids it is preferable to use protocol ET-pSPPS, according to some embodiments. Specifically, the protocol ET-pSPPS involves one cycle of MW coupling at 75° C. for 10 minutes using 3 equivalents of paa. According to some embodiments, the coupling protocol for the step of coupling the third paa in the peptide comprises the parameter of coupling duration in the range of 7.5 to 12.5 minutes. According to some embodiments, the coupling protocol for the step of coupling the third paa in the peptide comprises the parameter of coupling duration in the range of about 10 minutes. According to some embodiments, the coupling protocol for the step of coupling the third paa in the peptide comprises the parameter of 2-4 equivalents of paa. According to some embodiments, the coupling protocol for the step of coupling the third paa in the peptide comprises the parameter of about 3 equivalents of paa. According to some embodiments, the coupling protocol for the step of coupling the third paa in the peptide comprises the parameter of one coupling cycle.

For fourth phosphorylated amino acids it is preferable to the ET-pSPPS protocol 3 except for cases in which the fourth phosphorylated amino acid is introduced after already two phosphorylated threonine residues, according to some embodiments. In such case protocol DC-pSPPS is best used for the introduction of the fourth phosphorylated amino acids, according to some embodiments. Specifically, the protocol DC-pSPPS involves two cycles of MW coupling at 75° C. for 5 minutes using 3 equivalents of paa.

According to some embodiments, the coupling protocol for the step of coupling the fourth paa in the peptide comprises the parameter of coupling duration in the range of 2.5 to 7.5 minutes. According to some embodiments, the coupling protocol for the step of coupling the fourth paa in the peptide comprises the parameter of coupling duration of about 5 minutes. According to some embodiments, the coupling protocol for the step of coupling the fourth paa in the peptide comprises the parameter of 2-4 equivalents of paa. According to some embodiments, the coupling protocol for the step of coupling the fourth paa in the peptide comprises the parameter of about 3 equivalents of paa. According to some embodiments, the coupling protocol for the steps of coupling the fourth paa in the peptide comprises the parameter of one coupling cycle or two cycles.

For the fifth phosphorylated amino acids it is preferable to use the DC-pSPPS protocol except for cases in which the fifth phosphorylated amino acids is introduced after already three phosphorylated threonine residues. In such case protocol EBB-pSPPS is best used for the introduction of the fifth phosphorylated amino acids, according to some embodiments. Specifically, the protocol EBB-pSPPS involves two cycles of MW coupling at 75° C. for 5 minutes using 5 equivalents of paa.

According to some embodiments, the coupling protocol for the step of coupling the fifth paa in the peptide comprises the parameter of coupling duration in the range of 2.5 to 7.5 minutes. According to some embodiments, the coupling protocol for the step of coupling the fifth paa in the peptide comprises the parameter of coupling duration of about 5 minutes. According to some embodiments, the coupling protocol for the step of coupling the fifth paa in the peptide comprises the parameter of 3-5 equivalents of paa. According to some embodiments, the coupling protocol for the steps of coupling the fifth paa in the peptide comprises the parameter of two coupling cycles.

The sixth and seventh phosphorylated amino acids it is preferable to use using the protocol EBB-pSPPS, according to some embodiments.

According to some embodiments, each of the coupling protocols for the steps of coupling the sixth and seventh paas in the peptide comprises the parameter of coupling duration in the range of 2.5 to 7.5 minutes. According to some embodiments, each of the coupling protocols for the steps of coupling the sixth and seventh paas in the peptide comprises the parameter of coupling duration of about 5 minutes. According to some embodiments, each of the coupling protocols for the steps of coupling the sixth and seventh paas in the peptide comprises the parameter of 5-7 equivalents of paa. According to some embodiments, each of the coupling protocols for the steps of coupling the sixth and seventh paas in the peptide comprises the parameter of about 5 equivalents of paa. According to some embodiments, each of the coupling protocols for the steps of coupling the sixth and seventh paas in the peptide comprises the parameter of two coupling cycles.

Table 1 summarizes the conditions of each of the pSPPS, ET-pSPPS, DC-pSPPS and SEBB-pSPPS coupling protocols.

TABLE 1
conditions of coupling protocols

| Name | Heating | Duration | Equivalents | Cycles |
|---|---|---|---|---|
| pSPPS | MW 75° C. | 5 minutes | 3 | 1 |
| ET-pSPPS | MW 75° C. | 10 minutes | 3 | 1 |
| DC-pSPPS | MW 75° C. | 5 minutes | 3 | 2 |
| EBB-pSPPS | MW 75° C. | 5 minutes | 5 | 2 |

According to some embodiments, the first two phosphorylated residues in the peptide are coupled using 3 equivalents of phosphorylated amino acids for 5 minutes and one coupling cycle; the third and optionally forth phosphorylated amino acids in the peptide are couples using 3 equivalents of phosphorylated amino acids for 10 minutes and one coupling cycle; the fourth and/or fifth phosphorylated residues in the peptide are coupled using 3 equivalents of phosphorylated amino acids for 5 minutes and two coupling cycles; and the fifth and/or sixth and following phosphorylated residues in the peptide are coupled using 5 equivalents of phosphorylated amino acids for 5 minutes and two coupling cycles.

According to some embodiments, at least two coupling protocols differ in at least one parameter, selected from: at least 25% difference in coupling duration, at least 3 minutes difference in coupling duration, at least 20% in molar equivalents of the paa, and the number of coupling cycles. According to some embodiments, at least two coupling protocols differ in at least one parameter, selected from: at least 50% difference in coupling duration, at least 4 minutes difference in coupling duration, at least 33% in molar equivalents of the paa, and the number of coupling cycles.

According to some embodiments, at least two coupling protocols differ in at least one parameter, selected from: at least 75% difference in coupling duration, at least 5 minutes difference in coupling duration, at least 50% in molar equivalents of the paa, and the number of coupling cycles. According to some embodiments, at least three coupling protocols differ in at least one parameter, selected from: at least 25% difference in coupling duration, at least 3 minutes difference in coupling duration, at least 20% in molar equivalents of the paa, and the number of coupling cycles. According to some embodiments, at least three coupling protocols differ in at least one parameter, selected from: at least 50% difference in coupling duration, at least 4 minutes difference in coupling duration, at least 33% in molar equivalents of the paa, and the number of coupling cycles. According to some embodiments, at least three coupling protocols differ in at least one parameter, selected from: at least 75% difference in coupling duration, at least 5 minutes difference in coupling duration, at least 50% in molar equivalents of the paa, and the number of coupling cycles. According to some embodiments, at least four coupling protocols differ in at least one parameter, selected from: at least 25% difference in coupling duration, at least 3 minutes difference in coupling duration, at least 20% in molar equivalents of the paa, and the number of coupling cycles. According to some embodiments, at least four coupling protocols differ in at least one parameter, selected from: at least 50% difference in coupling duration, at least 4 minutes difference in coupling duration, at least 33% in molar equivalents of the paa, and the number of coupling cycles. According to some embodiments, at least four coupling protocols differ in at least one parameter, selected from: at least 75% difference in coupling duration, at least 5 minutes difference in coupling duration, at least 50% in molar equivalents of the paa, and the number of coupling cycles. According to some embodiments, at least two coupling protocols differ in at least 25% difference in coupling duration. According to some embodiments, at least two coupling protocols differ in at least 50% difference in coupling duration. According to some embodiments, at least two coupling protocols differ in at least 75% difference in coupling duration.

According to some embodiments, at least two coupling protocols differ in at least 3 minutes difference in coupling duration. According to some embodiments, at least two coupling protocols differ in at least 4 minutes difference in coupling duration. According to some embodiments, at least two coupling protocols differ in at least 5 minutes difference in coupling duration. According to some embodiments, at least two coupling protocols differ by at least 20% in molar equivalents of the paa. According to some embodiments, at least two coupling protocols differ by at least 33% in molar equivalents of the paa. According to some embodiments, at least two coupling protocols differ by at least 50% in molar equivalents of the paa.

According to some embodiments, at least two coupling protocols differ in number of coupling cycles.

According to some embodiments, the method comprises at least a first paa coupling step and a second paa coupling step, wherein the first paa coupling step comprises coupling a first paa for a first duration, wherein the second paa coupling step comprises coupling a second paa for a second duration, and wherein the second duration is at least 40% higher than the first duration.

According to some embodiments, the method comprises at least a first paa coupling step and a second paa coupling step, wherein the first paa coupling step comprises coupling a first paa for a first duration, wherein the second paa coupling step comprises coupling a second paa for a second duration, and wherein the second duration is at least 60% higher than the first duration.

According to some embodiments, the method comprises at least a first paa coupling step and a second paa coupling step, wherein the first paa coupling step comprises coupling a first paa for a first duration, wherein the second paa coupling step comprises coupling a second paa for a second duration, and wherein the second duration is at least 80% higher than the first duration.

According to some embodiments, the method comprises at least a first paa coupling step and a second paa coupling step, wherein the first paa coupling step comprises coupling a first paa for a first duration, wherein the second paa coupling step comprises coupling a second paa for a second duration, and wherein the second duration is at least 1 minute longer than the first duration.

According to some embodiments, the method comprises at least a first paa coupling step and a second paa coupling step, wherein the first paa coupling step comprises coupling a first paa for a first duration, wherein the second paa coupling step comprises coupling a second paa for a second duration, and wherein the second duration is at least 2 minutes longer than the first duration.

According to some embodiments, the method comprises at least a first paa coupling step and a second paa coupling step, wherein the first paa coupling step comprises coupling a first paa for a first duration, wherein the second paa coupling step comprises coupling a second paa for a second duration, and wherein the second duration is at least 4.5 minutes longer than the first duration.

According to some embodiments, the method comprises at least a first paa coupling step and a second paa coupling step, wherein the first paa coupling step comprises coupling a first paa using a first predetermined value of molar equivalents of the first paa, wherein the second paa coupling step comprises coupling a second paa using a second predetermined value of molar equivalents of the second paa, and wherein the second predetermined value is at least 33% larger than the second predetermined value.

According to some embodiments, the method comprises at least a first paa coupling step and a second paa coupling step, wherein the first paa coupling step comprises coupling a first paa using a first predetermined value of molar equivalents of the first paa, wherein the second paa coupling step comprises coupling a second paa using a second predetermined value of molar equivalents of the second paa, and wherein the second predetermined value is at least 40% larger than the second predetermined value.

According to some embodiments, the method comprises at least a first paa coupling step and a second paa coupling step, wherein the first paa coupling step comprises coupling a first paa using a first predetermined value of molar equivalents of the first paa, wherein the second paa coupling step comprises coupling a second paa using a second predetermined value of molar equivalents of the second paa, and wherein the second predetermined value is at least 60% larger than the second predetermined value.

According to some embodiments, the method comprises at least a first paa coupling step and a second paa coupling step, wherein the first paa coupling step comprises coupling a first paa a first number of coupling cycles, wherein the second paa coupling step comprises coupling a second paa a second number of coupling cycles, and wherein the second number of coupling cycles is larger than the first number of coupling cycles by at least 1. According to some embodiments, the first paa coupling step comprises coupling the first paa once and the second paa coupling step comprises coupling the second paa twice.

According to some embodiments, at least one protocol comprises a step of Fmoc removal, comprising contacting the with an amine at least once for a duration of 5-20 minutes at a temperature in the range of 10-80° C. According to some embodiments, at least one protocol comprises a step of Fmoc removal, comprising contacting the with an amine for a first contact at a temperature in the range of 20-30° C. for about 10 minutes, and for a second contact at a temperature in the range of 20-30° C. for about 15 minutes. According to some embodiments, the first contact precedes the second contact. According to some embodiments, the second contact precedes the first contact. According to some embodiments, the amine is piperidine. According to some embodiments, the Fmoc removal is performed in dimethylformamide solvent. According to some embodiments, the Fmoc removal is performed using MW irradiation.

According to some embodiments, at least two protocols each comprise a step of Fmoc removal, comprising contacting the with an amine at least once for a duration of 5-20 minutes at a temperature in the range of 10-80° C. According to some embodiments, at least two protocols each comprise a step of Fmoc removal, comprising contacting the with an amine for a first contact at a temperature in the range of 20-30° C. for about 10 minutes, and for a second contact at a temperature in the range of 20-30° C. for about 15 minutes. According to some embodiments, the first contact precedes the second contact. According to some embodiments, the second contact precedes the first contact. According to some embodiments, the amine is piperidine. According to some embodiments, the Fmoc removals are performed in dimethylformamide solvent. According to some embodiments, the Fmoc removals are performed using MW irradiation.

According to some embodiments, at least three protocols each comprise a step of Fmoc removal, comprising contacting the with an amine at least once for a duration of 5-20 minutes at a temperature in the range of 10-80° C. According to some embodiments, at least three protocols each comprise a step of Fmoc removal, comprising contacting the with an amine for a first contact at a temperature in the range of 20-30° C. for about 10 minutes, and for a second contact at a temperature in the range of 20-30° C. for about 15 minutes. According to some embodiments, the first contact precedes the second contact. According to some embodiments, the second contact precedes the first contact. According to some embodiments, the amine is piperidine. According to some embodiments, the Fmoc removals are performed in dimethylformamide solvent. According to some embodiments, the Fmoc removals are performed using MW irradiation.

According to some embodiments, each of the protocols comprise a step of Fmoc removal, comprising contacting the with an amine at least once for a duration of 5-20 minutes at a temperature in the range of 10-80° C. According to some embodiments, each of the protocols comprise a step of Fmoc removal, comprising contacting the with an amine for a first contact at a temperature in the range of 20-30° C. for about 10 minutes, and for a second contact at a temperature in the range of 20-30° C. for about 15 minutes. According to some embodiments, the first contact precedes the second contact. According to some embodiments, the second contact precedes the first contact. According to some embodiments, the amine is piperidine. According to some embodiments, the Fmoc removals are performed in dimethylformamide solvent. According to some embodiments, the Fmoc removals are performed using MW irradiation.

According to some embodiments, the present invention concerns phosphorylated peptides obtained and obtainable by the method disclosed herein. According to some embodiments, there are provided a multi-phosphorylated peptide synthesized according to the method of the current invention.

According to some embodiments, the peptide is selected from the group consisting of SEQ ID NO. 1, SEQ ID NO. 2, SEQ ID NO. 3, SEQ ID NO. 5, SEQ ID NO. 6, SEQ ID NO. 8, SEQ ID NO. 9, SEQ ID NO. 10, SEQ ID NO. 11 and SEQ ID NO. 12. According to some embodiments, the peptide is represented by SEQ ID NO. 1. According to some embodiments, the peptide is represented by SEQ ID NO. 2. According to some embodiments, the peptide is represented by SEQ ID NO. 3. According to some embodiments, the peptide is represented by SEQ ID NO. 5. According to some embodiments, the peptide is represented by SEQ ID NO. 6. According to some embodiments, the peptide is represented by SEQ ID NO. 8. According to some embodiments, the peptide is represented by SEQ ID NO. 9. According to some embodiments, the peptide is represented by SEQ ID NO. 10. According to some embodiments, the peptide is represented by SEQ ID NO. 11. According to some embodiments, the peptide is represented by SEQ ID NO. 12. Table 2 lists SEQ ID Nos. 1-12.

TABLE 2
SEQ ID Nos 1-12

| SEQ ID No | Sequence | PAAs |
|---|---|---|
| 1 | CDDEApSpTpTVpSKpTEpTpSQVAPA | 7 |
| 2 | pSKpTEpTpSQVAPA | 4 |
| 3 | DDEApSpTpTVpSKpTEpTpQVAPA | 7 |
| 4 | DDEASTTVpSKTETSQVA | 1 |
| 5 | DDEASpTpTVpSKTETSQVAPA | 3 |
| 6 | CDDEASpTpTVSKTETpSQVAPA | 3 |
| 7 | DDEApSTpTVSKpTETpSQVAPA | 4 |
| 8 | ASpTpTVpSKpTEpTSQVAPA | 5 |
| 9 | CDDEApSpTpTVpSKpTETSQVAPA | 5 |
| 10 | CDDEASpTpTVpSKpTEpTpSQVAPA | 6 |
| 11 | pTpTVpSKpTEpTpSQVAPA | 6 |
| 12 | pTEpTpSQVAPA | 3 |

The present invention also concerns peptides, that were previously not synthesized in their phosphorylated form having up to 20 amino acids and 3 to 8 phosphorylated Ser or Thr residues, synthesized using the methods disclosed.

The following non-limiting examples are presented in order to more fully illustrate certain embodiments of the invention. They should in no way, however, be construed as limiting the broad scope of the invention. One skilled in the art can readily devise many variations and modifications

### EXAMPLES

Materials and Methods. All solvents and reagents were used as supplied. (1-[Bis (dimethylamino)methylene]-1H-1,2,3-triazolo[4,5-b]pyridinium-3-oxidhexafluorophosphate) (HATU), Dimethylformamide (DMF) (peptide synthesis grade) and acetonitrile (HPLC grade) were purchased from Biolab Chemicals. diisopropylethylamine (DIPEA), piperidine,triisopropylsilane (TIS), D₂O and Trifluoroacetic acid (TFA) were purchased from Sigma-Aldrich, Israel. All the standard Fmoc-amino acids, Fmoc-Ser(HPO₃Bzl)-OH and Fmoc-Thr(HPO₃Bzl)-OH were purchased from Luxembourg Industries Limited, Israel. Rink amide MBHA polystyrene resinis purchased from GL Bio with 0.546 mmol/g loading.

Solid Phase Peptide Synthesis. Solid phase peptide synthesis was performed using a CEM-Discover Microwave assisted Peptide Synthesizer (CEM Corporation, Mathews, N.C.) using the Fmoc strategy. The maximum temperature for couplings was set at 75° C. with 25 W. The Phosphorylated peptides were synthesized typically on 0.1 mmol scale.

Coupling procedure for protocol 1 (MW-SPPS): Fmoc-protected amino acid (5 equivalents), 4.5 equivalents HATU and DIPEA (8 equivalents) were dissolved in DMF (6 mL). The mixture was allowed to activate for 5 min at 0° C. then added to the resin bearing free amine. The reaction mixture was then microwave-irradiated for 5 min at 75° C. The resin was allowed to cool to room temperature and then washed thoroughly with DMF. All the Fmoc deprotections were performed by treating the peptidyl-resin twice with 20% piperidine in DMF for 2 min and 4 min at 75° C. temperature, both using MW irradiation. The solid support was then washed thoroughly with DMF.

Coupling procedure for protocol 2 (pSPPS): Fmoc-protected amino acid (3 equivalents), 2.5 equivalents HATU and DIPEA (6 equivalents) were dissolved in DMF(3mL). The mixture was allowed to activate for 5 min at 0° C. then added to the resin bound peptide. The solid support was then microwave-irradiated for 5 min at 75° C. The resin was allowed to cool to room temperature then washed thoroughly with DMF. All the Fmoc deprotections were performed by treating the peptidyl-resin twice with 20% piperidine in DMF for 10 min and 15 min at room temperature without the use of MW. The solid support was then washed thoroughly with DMF. All the non-phosphorylatedamino acids were coupled using 5 equivalents of amino acids, 4.5 equivalents of HATU and 8 equivalents of DIPEA in 6 mLof DMF employing conditions from protocol 2.

Coupling procedure for protocol 3 (ET-pSPPS): Fmoc-protected amino acid (3 equivalents), 2.5 equivalents HATU and DIPEA (6 equivalents) were dissolved in DMF (3 mL). The mixture was allowed to activate for 5 min at 0° C. and treated with the resin bearing free amine. The solid support was then microwave-irradiated for 10 min at 75° C. The resin was allowed to cool to room temperature then washed thoroughly with DMF. All Fmoc deprotections were performed by treating the peptidyl-resin twice with 20% piperidine in DMF for 10 min and 15 min at room temperature without the use of MW. The solid support was then washed thoroughly with DMF. All the non-phosphorylated amino acids were coupled using 5 equivalents of amino acids, 4.5 equivalents of HATU and 8 equivalents of DIPEA in 6 mL of DMF employing the above conditions.

Coupling procedure for protocol 4 (DC-pSPPS): Fmoc-protected amino acid (3 equivalents), 2.5 equivalents HATU and DIPEA (6 equivalents) were dissolved in DMF (3 mL). The mixture was activated at 0° C. for 5 min. The activated amino acid solution was then added to the resin bound peptide. The double coupling was performed at 75° C. for 5 min each time using 3equivalents of fresh phosphorylated amino acid in each cycle. The resin was washed thoroughly with DMF and the completion of coupling was checked by Kaiser test and HPLC-MS analysis performed after cleavage from the solid support. All the Fmoc deprotections were performed by treating the peptidyl-resin twice with 20% piperidine in DMF for 10 min and 15 min at room temperature without the use of MW. The solid support was then washed thoroughly with DMF. All the non-phosphorylated amino acids were coupled using 5 equivalents of amino acids, 4.5 equivalents of HATU and 8 equivalents of DIPEA in 6 mL of DMF employing the conditions from protocol 4.

Coupling procedure for protocol 5 (EBB-pSPPS): Fmoc-protected amino acid (5 equivalents), 4.5 equivalents HATU and DIPEA (8 equivalents) were dissolved in DMF(6 ml).The mixture was allowed to activate for 5 min at 0° C. The activated amino acid solution was then added to the resin with free amine. The 10 min coupling was performed in two cycles using 5 equivalents of fresh phosphorylated amino acid in each cycle under microwave-irradiation at 75° C. The resin was allowed to cool to room temperature then washed thoroughly with DMF. The reaction was monitored by Kaiser test. All the Fmoc deprotections were performed by treating the peptidyl resin twice with 20% piperidine in DMF for 10 min and 15 min at room temperature without the use of MW. The solid support was then washed thoroughly with DMF. All the non-phopshorylated amino acid residues were also incorporated employing the same conditions.

Room temperature Fmoc deprotection: The Fmoc-peptidyl-resin was treated twice with 20% piperidine in DMF (6 mL) for 10 min and 15 min at room temperature. The solid support was then washed thoroughly with DMF.

Microwave Fmoc deprotection: The resin bound Fmoc-peptide was treated twice with 20% piperidine in DMF (6 mL) for 2 min and 4 min at 75° C. under microwave irradiation. The resulting deprotected peptide was allowed to cool to room temperature and thoroughly washed with DMF.

Cleavage from the resin: A freshly prepared solution (5 mL) of trifluoroacetic acid (TFA)/triisopropylsilane (TIS)/TDW/ethane dithiol (EDT) (94:1:2.5:2.5) was cooled to 0° C. and added to 200 mg resin-bound peptide. The mixture was shaken at room temperature according to the times given below for each sequence. Then, the solid support was separated by filtration. The TFA was removed under nitrogen atmosphere and the peptide was precipitated by gradual addition of ice-cold ether to the mixture. The solution was centrifuged and the peptide washed twice with ether. A minimum volume of a 1:2 ACN/TDW mixture was used to dissolve the crude peptide, which was then lyophilized before HPLC purification and MS analysis.

RP-HPLC analysis: The crude phosphorylated peptides were analyzed by Merck Hitachi HPLC with a reverse-phase Agilent analytical column (eclipse XDB-AgilentC18, 4.6×150 mm; 5 μm) using a linear gradient of 1-30% Acetonitrile in water over 30 min with 0.1% TFA.

RP-HPLC purification: The crude peptides were purified by Merck Hitachi HPLC with a reverse-phase C18 semi prep column (Merck purospher STAR Rp-18e; 5 μm) with flow rate of 4.5 mL/min using a liner gradient of 2-40% acetonitrile in water, over 40 min with 0.1% TFA.

UPLC analysis: Pure phosphorylated peptides were characterized by analytical reversed-phaseAcquity UPLC H-Class with the UV detection (220 nm and 280 nm) using a Waters™ XSelect C18 column (3.5 μm, 130 Å, 4.6×150 mm). The flow rate was set to 1 mL/min using a linear gradient of 1-30% of acetonitrile in water, 0.1% TFA in 30 minutes.

NMR Analysis: ³¹P NMR spectra of all the phosphopeptides were recorded in D₂O using BBO-5 mm probe on Bruckeradvance-II 202.4 MHz instrument.

Mass spectrometry: Phosphorylated peptides were characterized by Electrospray ionization MS on LCQ Fleet Ion Trap mass spectrometer instrument (Thermo Scientific). Peptides masses were calculated from the experimental mass to charge (m/z) ratios from all of the observed multiply charged species of a peptide. Deconvolution of the experimental MS data was performed with the help of the MagTran v1.03 software.

### Example 1

Synthesis and characterization of Peptide 1 (SEQ ID NO. 1): Peptide 1 was synthesized following the sequence of protocols described in FIG. 3A. The peptide was fully cleaved from the solid support following 8 hours incubation in the TFA mixture as described above. FIG. 3B illustrates an RP-UPLC Chromatogram of peptide 1. FIG. 3C illustrates an . deconvoluted mass spectra of peptide 1 Obs.2598.46 Da; Calc. 2599.02 Da. FIG. 3D illustrates a ³¹P NMR spectra of phosphopeptide 1 (SEQ ID NO. 1).

### Example 2

Synthesis and characterization of Peptide 2 (SEQ ID NO. 2): Peptide 2 was synthesized following the sequence of protocols described in FIG. 4A. The peptide was fully cleaved from the solid support following 5 hours incubation in the TFA mixture as described above.

FIG. 4A illustrates the sequence of protocols used in the synthesis of peptide 2. FIG. 4B illustrates RP-UPLC Chromatogram of peptide 2 (conditions: flow rate: 1 mL/min; Acquity UPLC H-class Xselect C18 column, 4.6×150 mm; 3.5 μm; gradient: ACN: H₂O 1-30% ACN in 30 min; 220 nm). FIG. 4C illustrates deconvoluted mass spectra of Peptide 2. Obs. 1436.84 Da; Calc.1437.13 Da.

### Example 3

Synthesis and characterization of Peptide 3 (SEQ ID NO. 3): Peptide 3 was synthesized following the sequence of protocols described in FIG. 5A. The peptide was fully cleaved from the solid support following 8 hours incubation in the TFA mixture as described above. FIG. 5A illustrates the sequence of protocols used in the synthesis of Peptide 3. FIG. 5B illustrates an RP-UPLC Chromatogram of Peptide 3 (conditions: flow rate: 1 mL/min; Acquity UPLC H-class Xselect C18 column, 4.6×150 mm; 3.5 μtm; gradient: ACN: H2O 1-30% ACN in 30 min; 220 nm). FIG. 5C illustrates deconvoluted mass spectra of Peptide 3. Obs. 2496.22 Da; Calc.2495.87 Da.

### Example 4

Synthesis and characterization of Peptide 4 (SEQ ID NO. 4): Peptide 4 was synthesized following the sequence of protocols described in FIG. 6A. The peptide was fully cleaved from the solid support following 3 hours incubation in the TFA mixture as described above. FIG. 6A illustrates the sequence of protocols used in the synthesis of Peptide 4. FIG. 6B illustrates an RP-UPLC Chromatogram of Peptide 4 (conditions: flow rate: 1 mL/min; Acquity UPLC H-class Xselect C18 column, 4.6×150 mm; 3.5 μm; gradient: ACN: H₂O 1-30% ACN in 30 min; 220 nm). FIG. 6C illustrates deconvoluted mass spectra of Peptide 4. Obs.2015.51 Da; Calc.2015.98 Da. FIG. 6D illustrates ³¹P NMR spectra of Peptide 4.

### Example 5

Synthesis and characterization of Peptide 5 (SEQ ID NO. 5): Peptide 5 was synthesized following the sequence of protocols described in FIG. 7A. The peptide was fully cleaved from the solid support following 4.5 hours incubation in the TFA mixture as described above. FIG. 7A illustrates the sequence of protocols used in the synthesis of Peptide 5. FIG. 7B illustrates an RP-UPLC Chromatogram of Peptide 5 (conditions: flow rate: 1 mL/min; Acquity UPLC H-class Xselect C18 column, 4.6×150 mm; 3.5 μm; gradient: ACN: H₂O 1-30% ACN in 30 min; 220 nm). FIG. 7C illustrates deconvoluted mass spectra of Peptide 5. Obs.2174.87 Da; Calc.2175.94 Da. FIG. 7D illustrates ³¹P NMR spectra of Peptide 5.

### Example 6

Synthesis and characterization of Peptide 6 (SEQ ID NO. 6): Peptide 6 was synthesized following the sequence of protocols described in FIG. 8A. The peptide was fully cleaved from the solid support following 4.5 hours incubation in the TFA mixture as described above. FIG. 8A illustrates the sequence of protocols used in the synthesis of Peptide 6. FIG. 8B illustrates an RP-HPLC Chromatogram of Peptide 6 (conditions: flow rate: 1 mL/min; Acquity UPLC H-class Xselect C18 column, 4.6×150 mm; 3.5 μm; gradient: ACN: H₂O 1-30% ACN in 30 min; 220 nm). FIG. 8C illustrates de convoluted mass spectra of Peptide 6 Obs.2278.44 Da; Calc. 2279.08 Da.

### Example 7

Synthesis and characterization of Peptide 7 (SEQ ID NO. 7): Peptide 7 was synthesized following the sequence of protocols described in FIG. 9A. The peptide was fully cleaved from the solid support following 5.5 hours incubation in the TFA mixture as described above. FIG. 9A illustrates the sequence of protocols used in the synthesis of Peptide 7. FIG. 9B illustrates an RP-UPLC Chromatogram of Peptide 7 (conditions: flow rate: 1 mL/min; Acquity UPLC H-class Xselect C18 column, 4.6×150 mm; 3.5 μm; gradient: ACN: H₂O 1-30% ACN in 30 min; 220 nm). FIG. 9C illustrates deconvoluted mass spectra of Peptide 7. Obs.2255.63 Da; Calc.2255.92 Da. FIG. 9D illustrates ³¹P NMR spectra of Peptide 7.

### Example 8

Synthesis and characterization of Peptide 8 (SEQ ID NO. 8): Peptide 8 was synthesized following the sequence of protocols described in FIG. 10A. The peptide was fully cleaved from the solid support following 6 hours incubation in the TFA mixture as described above. FIG. 10A illustrates the sequence of protocols used in the synthesis of Peptide 8. FIG. 10B illustrates an RP-UPLC Chromatogram of Peptide 8 (conditions: flow rate: 1 mL/min; Acquity UPLC H-class Xselect C18 column, 4.6×150 mm; 3.5 μm; gradient: ACN: H₂O 1-30% ACN in 30 min; 220 nm). FIG. 10C illustrates deconvoluted mass spectra of Peptide 8. Obs.1976.35 Da; Calc.1976.62 Da. FIG. 10D illustrates ³¹P NMR spectra of Peptide 8.

### Example 9

Synthesis and characterization of Peptide 9 (SEQ ID NO. 9): Peptide 9 was synthesized following the sequence of protocols described in FIG. 11A. The peptide was fully cleaved from the solid support following 6.5 hours incubation in the TFA mixture as described above. FIG. 11A illustrates the sequence of protocols used in the synthesis of Peptide 9. FIG. 11B illustrates an RP-UPLC Chromatogram of Peptide 9 (conditions: flow rate: 1 mL/min; Acquity UPLC H-class Xselect C18 column, 4.6×150 mm; 3.5 μm; gradient: ACN: H₂O 1-30% ACN in 30 min; 220 nm). FIG. 11C illustrates deconvolutedmass spectra of Peptide 9. Obs.2437.92 Da; Calc.2439.05Da. FIG. 11D illustrates ³¹P NMR spectra of Peptide 9.

### Example 10

Synthesis and characterization of Peptide 10 (SEQ ID NO. 10): Peptide 10 was synthesized following the sequence of protocols described in FIG. 12A. The peptide was fully cleaved from the solid support following 7.5 hours incubation in the TFA mixture as described above. FIG. 12A illustrates the sequence of protocols used in the synthesis of Peptide 10. FIG. 12B illustrates an RP-UPLC Chromatogram of Peptide 10 (conditions: flow rate: 1 mL/min; Acquity UPLC H-class Xselect C18 column, 4.6×150 mm; 3.5 μm; gradient: ACN: H₂O 1-30% ACN in 30 min; 220 nm). FIG. 12C illustrates de convoluted mass spectra of Peptide 10. Obs.2518.56 Da; Calc.2519.03 Da. FIG. 12D illustrates ³¹P NMR spectra of Peptide 10.

### Example 11

Synthesis and characterization of Peptide 11 (SEQ ID NO. 11): Peptid 11 was synthesized following the sequence of protocols described in FIG. 13A. The peptide was fully cleaved from the solid support following 7 hours incubation in the TFA mixture as described above. FIG. 13A illustrates the sequence of protocols used in the synthesis of Peptide 11. FIG. 13B illustrates an RP-UPLC Chromatogram of Peptide 11 (conditions: flow rate: 1 mL/min; Acquity UPLC H-class Xselect C18 column, 4.6×150 mm; 3.5 μm; gradient: ACN: H₂O 1-30% ACN in 30 min; 220 nm). FIG. 13C illustrates deconvoluted mass spectra of Peptide 11. Obs.1897.70 Da; Calc. 1898.44 Da.

Conclusion and Remarks:

The synthesis of the Rho340-348 triphosphopeptide (pTEpTpSQVAPA—SEQ ID NO. 12) was first attempted using the previously reported protocols.⁴² HPLC analysis of the resulting peptide indicated that the desired product was obtained only in low crude purity (30%, FIG. 14A). Attempts to synthesize the tetra phosphorylated peptide (Rho338-348, pSKpTEpTpSQVAPA—SEQ ID NO. 2) employing the same reported conditions resulted in sharp decrease in the peptide purity (10%, FIG. 2B). FIG. 14A depicts an RP-HPLC chromatogram of the peptide SEQ ID NO. 12 bearing three phosphorylations, synthesized employing previously reported conditions⁴²; FIG. 14B depicts an RP-HPLC chromatogram of the tetra phosphorylated peptide SEQ ID NO. 2 synthesized using the same reported conditions; FIG. 14C depicts an RP-HPLC chromatogram of the tetra phosphorylated peptide SEQ ID NO. 2 synthesized employing the method of the current invention as detailed in FIG. 4A.

These results suggested that the synthesis of Rho330-348 derived peptides with more than three phosphorylations is problematic using the currently available Fmoc-SPPS protocols. To overcome this limitation, a new strategy was developed for enabling the synthesis of Rho330-348 peptides with all the seven phosphorylation sites using MW assisted Fmoc-SPPS. The extended coupling protocols previously used in difficult coupling steps resulted in longer reaction times and excess usage of reagents and solvents and thus are not cost effective. This is an important factor in the synthesis of multi phosphorylated peptides because protected phosphorylated amino acids are highly expensive. The method of the current invention provides efficient coupling yields without using large amounts of phosphorylated amino acids.

Seven phosphorylated Rho330-348 derived peptide (Peptide 1—SEQ ID NO. 1) were prepared and monitored using the progress using Kaiser test (49-50) and/or HPLC-MS analysis. The commercially available mono-benzyl protected amino acids Fmoc-Ser(HPO₃Bzl)-OH and Fmoc-Thr(HPO₃Bzl)-OH were incorporated using MW assisted coupling conditions. The selected activating system was HATU/DIEA, which was reported to be efficient for phosphopeptide synthesis.³⁷ The detailed conditions and protocols used in each step of the synthesis of Peptide 1 are described above and are summarized in FIG. 3A.

The synthesis of Peptide 1 up to Gln344 was performed using different MW SPPS protocols. Coupling was performed using HATU/DIEA in DMF under microwave at 25W with a set temperature of 75° C. for 5 min. Fmoc-deprotection was performed twice with 20% piperidine/DMF under microwave (2 and 4 min) at 75° C. (Protocol 1—MW SPPS). For the insertion of the first two phosphorylated residues (Thr342 and Ser343) three equivalents of phosphorylated amino acids were used, activated with HATU/DIEA, and coupling at 75° C. for 5 min (protocol 2—pSPPS). After each coupling, the solid support was washed thoroughly with DMF (5×5 mL) to ensure that the temperature is below 30° C. before the deprotection reaction. The MW assisted Fmoc-deprotection strategy was replaced by the standard SPPS protocol at room temperature (twice for 10 and 15 min) to avoid the β-elimination side reaction (protocol 2—pSPPS).

The results showed that the pSPPS protocol is efficient for the insertion of up to two phosphorylated amino acids. Applying the pSPPS protocol for the insertion of Glu341 resulted in a coupling that did not reach completion. Extending the coupling time to 10 minutes allowed for the complete coupling of Glu341 (Protocol 3 ET-pSPPS). The analysis confirmed that following the ET-pSPPS protoco was crucial also for the insertion of pThr340, Lys339 and pSer338. RP-HPLC analysis indicated that the purity of the desired tetra phosphorylated peptide 2 (SEQ ID NO. 2) in its crude form was 80%, see FIG. 14C and FIG. 4B-C).

The results also showed that a combination of the protocols MW-SPPS, pSPPS and ET-pSPPS is highly efficient for the synthesis of a peptide with four phosphorylations on adjacent residues (SEQ ID NO. 2). This strategy provided a vast improvement in yield and purity compared to the known protocol (compare FIGS. 14B and 14C). ET-pSPPS protocol was also successfully used for the insertion of Val 337. However, the introduction of the fifth phosphorylated amino acid (pThr336) by employing the ET-pSPPS protocol resulted in incomplete couplings even after extending the coupling time to 15 minutes (monitored by Kaiser test). The remaining part of Rho330-348phosphopeptide contains a very challenging sequence of three successive phosphorylated amino acids (pSer334-pThr335-pThr336), which is even more challenging within a peptide sequence that already contains four phosphorylated amino acids. In addition, couplings of pThr are more difficult compared to pSer and may lead to deletion sequences. To overcome these difficulties, we employed repeating coupling cycles (double coupling) strategy. MW assisted coupling for 10 min using 3 equivalents of amino acid in each cycle was applied twice for the introduction of pThr336 (DC-pSPPS protocol). The results indicated a full coupling of the fifth phosphorylated amino acid (pThr336).

After the successful insertion of pThr336, applying the DC-pSPPS protocol for the insertion of pThr335 resulted in only 40-50% coupling even when reaction time was increased to 15 minutes. Without wishing to be bound by any theory or mechanism of action, it is hypothesized that this since the coupling of two successive protected pThr is extremely challenging. This is probably due to the presence of the bulky benzyl groups and Thr is a β-branched amino acid, which might results in low accessibility to the nucleophilic amine. As the use of 3 equivalents of amino acid proved insufficient for the introduction of pThr335 and pSer334, the equivalents of amino acid used in each cycle was increased from three to five. These coupling conditions resulted in successful insertion of both pThr335 and pSer334 (EBB-pSPPS protocol).

Altogether, the use of a strong activator, 5 equivalents of pAAs in each cycle, a temperature of 75° C. for 10 min and the MW couplings overcame the steric hindrance that results from the presence of multiple neighboring phosphorylated amino acids and promoted the insertion of pThr335 pSer334 in a most efficient manner using Protocol 5. The introduction of Ala333 was also possible only using the EBB-pSPPS protocol. Coupling of the rest of the amino acids was performed successfully using the ET-pSPPS protocol without any observed difficulties (FIG. 3A). After the completion of the solid phase synthesis, peptide 1 (SEQ ID NO. 1) was cleaved from the solid support using TFA:TIS:H₂O:EDT (9.4:1:2.5:2.5, v/v/v/v). Initial attempts to cleave Peptide 1 from the solid support resulted in incomplete removal of all the benzyl protecting groups form the phosphates. Fully deprotected phosphorylated Peptide 1 was obtained when the cleavage was extended to 8 h at room temperature. The crude peptide 1 was purified by preparative HPLC. The isolated pure peptide was characterized by MS, HPLC and ³¹P NMR (FIGS. 3B-D).

A series of multi-phosphorylated peptides derived from Rho330-348 with various phosphorylation patterns (SEQ ID Nos. 2-11) were prepared. All the multi-phosphorylated peptides were synthesized on solid support according to the protocols listed above. The peptides were cleaved from the resin using TFA:TIS:H₂O (95:2.5:2.5) or TFA:TIS:H₂O:EDT (94:1:2.5:2.5, if cysteine was present in the sequence) at room temperature. The cleavage time required for the complete release of all the benzyl protecting groups from the phosphor-amino acids depended on the number of phosphorylation sites. Hence, cleavage duration was extended according to the degree of phosphorylation.

The crude peptides were analyzed using analytical RP-HPLC/MS and purified using preparative RP-HPLC (FIGS. 4B-13B and 4C-13C). The isolated phosphopeptides were analyzed further using ESI-MS, ³¹P NMR and their purity was confirmed using UPLC analysis. The overlay of UPLC chromatograms of the phosphorylated peptides with 1-7 phosphorylation sites is shown in FIG. 15. A decrease in retention time was observed with the increasing amount of phosphorylated amino acids.

Peptides 1-11 (SEQ ID Nos. 1-11 respectively) were synthesized using a specific combination of protocols for each peptide (Examples 1-11). No single protocol proved efficient or economical to introduce all the pAAs. Without wishing to be bound by any theory or mechanism of action, the type of protocols used in each step of the synthesis was highly dependent on the number and type of phosphorylated amino acids in the sequence. In all the multi phosphorylated peptides, the two C-terminal phosphorylated amino acids can be coupled using the pSPPS protocol irrespective of their type and proximity. The third phosphorylated serine can be introduced successfully using the ET-pSPPS protocol in all cases. This shows that for up to three pAAs, 3 equivalents of pAA is sufficient for getting complete coupling. The sixth and seventh pAAs can be introduced only using the EBB-pSPPS protocol. This indicates that only in these cases significant excess of pAA and double coupling are required. The protocol required for the introduction of the fourth and fifth phosphorylated amino acids depends on the phosphorylation pattern of each peptide. While in some cases the Et-pSPPS protocol can be used for the introduction of the fourth phosphorylated amino acid, in other cases the fourth pAA can be introduced only using the DC-pSPPS protocol, which uses double coupling. This depends specifically on the difference between pThr and pSer. When the fourth pAA is a serine, the Et-pSPPS protocol proved efficient enough (peptides 1,2,3,7,10,11—SEQ ID NOs. 2,3,7,10,11 respectively). However, when the fourth pAA is a threonine that is introduced into a peptide that already has two pThr residues, the ET-pSPPS protocol proved insufficient and the DC-pSPPS protocol was necessary to enable efficient coupling (peptides 8 and 9, SEQ ID NOs 8 and 9 respectively). Apparently, this indicates that crowding of bulky β-branched Thr with benzyl protection slows down the coupling, an effect that is less significant for pSer.

The multi-protocol method of the current invention is essential to the production of the elusive synthetic multi phosphorylated peptides because the difficulties in the coupling depend on the proximity, type and the number of the phosphorylated amino acids in the peptide. While previous reports^{35,36,42} used a constant set of conditions that required large quantities of phosphorylated amino acids for each coupling, the current method is much more economical.

The foregoing description of the specific embodiments, will so fully reveal the general nature of the invention that others can, by applying current knowledge, readily modify and/or adapt for various applications such specific embodiments, without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

### REFERENCES

(1) Cohen, P. Nature 1982, 296, 613.

(2) Cohen, P. Seikagaku 1982, 54, 1011.

(3) Crosio, C.; Fimia, G. M.; Loury, R.; Kimura, M.; Okano, Y.; Zhou, H. Y.; Sen, S.; Allis, C. D.; Sassone-Corsi, P. Mol Cell Biol 2002, 22, 874.

(4) Liu, C. M.; Li, Y. M.; Semenov, M.; Han, C.; Baeg, G. H.; Tan, Y.; Zhang, Z. H.; Lin, X. H.; He, X. Cell 2002, 108, 837.

(5) Cohen, P. Eur J Biochem 2001, 268, 5001.

(6) Haycock, J. W.; Lew, J. Y.; Garcia-Espana, A.; Lee, K. Y.; Harada, K.; Meller, E.; Goldstein, M. J Neurochem 1998, 71, 1670.

(7) Songyang, Z.; Shoelson, S. E.; Mcglade, J.; Olivier, P.; Pawson, T.; Bustelo, X. R.; Barbacid, M.; Sabe, H.; Hanafusa, H.; Yi, T.; Ren, R.; Baltimore, D.; Ratnofsky, S.; Feldman, R. A.; Cantley, L. C. Mol Cell Biol 1994, 14, 2777.

(8) Waksman, G.; Shoelson, S. E.; Pant, N.; Cowburn, D.; Kuriyan, J. Cell 1993, 72, 779.

(9) Flick, M. B.; Sapi, E.; Perrotta, P. L.; Maher, M. G.; Halaban, R.; Carter, D.; Kacinski, B. M. Oncogene 1997, 14, 2553.

(10) Kuropka, B.; Witte, A.; Sticht, J.; Waldt, N.; Majkut, P.; Hackenberger, C. P. R.; Schraven, B.; Krause, E.; Kliche, S.; Freund, C. Mol Cell Proteomics 2015, 14, 2961.

(11) Kapuy, O.; Barik, D.; Sananes, M. R. D.; Tyson, J. J.; Novak, B. Prog Biophys Mol Bio 2009, 100, 47.

(12) Roach, P. J. J Biol Chem 1991, 266, 14139.

(13) Cohen, P. Trends Biochem Sci 2000, 25, 596.

(14) Attard, T. J.; O'Brien-Simpson, N.; Reynolds, E. C. Int J Pept Res Ther 2007, 13, 447.

(15) Bannwarth, W.; Kung, E. Tetrahedron Lett 1989, 30, 4219.

(16) Mora, N.; Lacombe, J. M.; Pavia, A. A. Tetrahedron Lett 1993, 34, 2461.

(17) Paquet, A.; Johns, M. Int J Pept Prot Res 1990, 36, 97.

(18) Tong, G.; Perich, J. W.; Johns, R. B. Tetrahedron Lett 1990, 31, 3759.

(19) Tsukamoto, M.; Kato, R.; Ishiguro, K.; Uchida, T.; Sato, K. Tetrahedron Lett 1991, 32, 7083.

(20) Wakamiya, T.; Saruta, K.; Kusumoto, S.; Nakajima, K.; Yoshizawakumagaye, K.; Imajohohmi, S.; Kanegasaki, S. Chem Lett 1993, 1401.

(21) Qvit, N.; Hatzubai, A.; Shalev, D. E.; Friedler, A.; Ben-Neriah, Y.; Gilon, C. Biopolymers 2009, 91, 157.

(22) Bertran-Vicente, J.; Serwa, R. A.; Schumann, M.; Schmieder, P.; Krause, E.; Hackenberger, C. P. R. J Am Chem Soc 2014, 136, 13622.

(23) Staerkaer, G.; Jakobsen, M. H.; Olsen, C. E.; Holm, A. Tetrahedron Lett 1991, 32, 5389.

(24) Kitas, E. A.; Knorr, R.; Trzeciak, A.; Bannwarth, W. Helv Chim Acta 1991, 74, 1314.

(25) Bannwarth, W.; Kung, E.; Vorherr, T. Bioorg Med Chem Lett 1996, 6, 2141.

(26) Attard, T. J.; Reynolds, E. C.; Perich, J. W. Org Biomol Chem 2007, 5, 664.

(27) Perich, J. W.; Reynolds, E. C. Int J Pept Prot Res 1991, 37, 572.

(28) Bonewald, L. F.; Bibbs, L.; Kates, S. A.; Khatri, A.; McMurray, J. S.; Medzihradszky, K. F.; Weintraub, S. T. J Pept Res 1999, 53, 161.

(29) Chao, H. G.; Bernatowicz, M. S.; Reiss, P. D.; Matsueda, G. R. J Org Chem 1994, 59, 6687.

(30) Alewood, P. F.; Perich, J. W.; Johns, R. B. Aust J Chem 1984, 37, 429.

(31) Alewood, P. F.; Perich, J. W.; Johns, R. B. Tetrahedron Lett 1984, 25, 987.

(32) Otvos, L.; Elekes, I.; Lee, V. M. Y. Int J Pept Prot Res 1989, 34, 129.

(33) Wakamiya, T.; Saruta, K.; Yasuoka, J.; Kusumoto, S. Chem Lett 1994, 1099.

(34) Vorherr, T.; Bannwarth, W. Bioorg Med Chem Lett 1995, 5, 2661.

(35) Hong, A.; Zhou, Y.; Diwu, Z. Chim Oggi 2003, 21, 57.

(36) O'Brien-Simpson, N. M.; Attard, T. J.; Loganathan, A.; Huq, N. L.; Cross, K. J.; Riley, P. F.; Reynolds, E. C. Int J Pept Res Ther 2007, 13, 469.

(37) Perich, J. W.; Ede, N. J.; Eagle, S.; Bray, A. M. Lett Pept Sci 1999, 6, 91.

(38) Pedersen, S. L.; Tofteng, A. P.; Malik, L.; Jensen, K. J. Chem Soc Rev 2012, 41, 1826.

(39) Attard, T. J.; O'Brien-Simpson, N. M.; Reynolds, E. C. Int J Pept Res Ther 2009, 15, 69.

(40) Brandt, M.; Gammeltoft, S.; Jensen, K. J. Int J Pept Res Ther 2006, 12, 349.

(41) Qvit, N. Chem Biol Drug Des 2015, 85, 300.

(42) Harris, P. W. R.; Williams, G. M.; Shepherd, P.; Brimble, M. A. Int J Pept Res Ther 2008, 14, 387.

(43) Puig, J.; Arendt, A.; Tomson, F. L.; Abdulaeva, G.; Miller, R.; Hargrave, P. A.; Mcdowell, J. H. Febs Lett 1995, 362, 185.

(44) Kisselev, O. G.; Downs, M. A.; McDowell, J. H.; Hargrave, P. A. J Biol Chem 2004, 279, 51203.

(45) Arendt, A.; McDowell, J. H.; Abdulaeva, G.; Hargrave, P. A. Protein Peptide Lett 1996, 3, 361.

(46) Maeda, S.; Sun, D. W.; Singhal, A.; Foggetta, M.; Schmid, G.; Standfuss, J.; Hennig, M.; Dawson, R. J. P.; Veprintsev, D. B.; Schertler, G. F. X. Plos One 2014, 9.

(47) Mcdowell, J. H.; Nawrocki, J. P.; Hargrave, P. A. Biochemistry-Us 1993, 32, 4968.

(48) Mcdowell, J. H.; Nawrocki, J. P.; Hargrave, P. A. Invest Ophth Vis Sci 1993, 34, 1324.

(49) Kaiser, E.; Colescot.Rl; Bossinge.Cd; Cook, P. I. Anal Biochem 1970, 34, 595.

(50) Sarin, V. K.; Kent, S. B. H.; Tam, J. P.; Merrifield, R. B. Anal Biochem 1981, 117, 147.

1. A method of Fmoc-solid phase synthesis of a peptide that comprises at least 3 phosphorylated amino acid (paa) residues, wherein at least one of the paa residues is a phosphorylated threonine (p-Thr) residue and wherein at least two of the paa residues are adjacent, the method comprising separately coupling at least 3 paas, wherein each paa coupling step comprises a coupling protocol, each coupling protocol comprises the parameters of: coupling duration, molar equivalents of paa and number of coupling cycles, wherein at least one of the paa coupling steps is microwave assisted at a temperature of 60° C. to 85° C., and wherein at least two coupling protocols differ in at least one of the parameters.

2. The method of claim 1, wherein at least three couplings differ in at least one of the parameters.

3. The method of claim 1, wherein the peptide consists of 10-25 amino acid residues.

4. The method of claim 1, wherein the peptide comprises 3 to 7 paa residues.

5. The method of claim 1, wherein the peptide comprises at least two p-Thr residues.

6. The method of claim 1, wherein the peptide comprises at least two p-Thr residues and at least two phosphorylated Serine (p-Ser) residues.

7. The method of claim 1, wherein each of the coupling steps is microwave assisted at a temperature of 60° C. to 85° C.

8. The method of claim 1, wherein microwave assisted coupling is performed at about 75° C.

9. The method of claim 1, wherein the peptide comprises at least two sub-sequences, wherein each sub-sequence comprises at least two adjacent paa residues, selected from p-Thr-p-Ser, p-Ser-p-Thr and p-Thr-p-Thr.

10. The method of claim 1, wherein each coupling protocol comprises the parameters: coupling duration between 5 to 20 minutes, molar equivalents of paa between 3 and 6, and the number of coupling cycles between 1 to 3.

11. The method of claim 1, wherein each coupling protocol comprises a step of Fmoc removal, comprising contacting the with an amine at least once for a duration of 5-20 minutes at a temperature in the range of 10-80° C.

12. The method of claim 1, wherein:
the first two phosphorylated residues in the peptide are coupled using 3 equivalents of phosphorylated amino acids for 5 minutes and one coupling cycle;
the third and optionally forth phosphorylated amino acids in the peptide are couples using 3 equivalents of phosphorylated amino acids for 10 minutes and one coupling cycle;
the fourth and/or fifth phosphorylated residues in the peptide are coupled using 3 equivalents of phosphorylated amino acids for 5 minutes and two coupling cycles; and
the fifth and/or sixth and following phosphorylated residues in the peptide are coupled using 5 equivalents of phosphorylated amino acids for 5 minutes and two coupling cycles.

13. The method of claim 1, wherein the at least two coupling protocols differ in at least one parameter, selected from: at least 50% difference in coupling duration, at least 3 minutes difference in coupling duration, at least 33% in molar equivalents of the paa, and the number of coupling cycles.

14. The method of claim 1, comprising at least a first paa coupling step and a second paa coupling step, wherein the first paa coupling step comprises coupling a first paa for a first duration, wherein the second paa coupling step comprises coupling a second paa for a second duration, and wherein the second duration is at least 50% higher than the first duration.

15. The method of claim 1, comprising at least a first paa coupling step and a second paa coupling step, wherein the first paa coupling step comprises coupling a first paa for a first duration, wherein the second paa coupling step comprises coupling a second paa for a second duration, and wherein the second duration is at least 3 minutes longer than the first duration.

16. The method of claim 1, comprising at least a first paa coupling step and a second paa coupling step, wherein the first paa coupling step comprises coupling a first paa using a first predetermined value of molar equivalents of the first paa, wherein the second paa coupling step comprises coupling a second paa using a second predetermined value of molar equivalents of the second paa, and wherein the second predetermined value is at least 33% larger than the second predetermined value.

17. The method of claim 1, comprising at least a first paa coupling step and a second paa coupling step, wherein the first paa coupling step comprises coupling a first paa a first number of coupling cycles, wherein the second paa coupling step comprises coupling a second paa a second number of coupling cycles, and wherein the second number of coupling cycles is larger than the first number of coupling cycles by at least 1.

18. The method of claim 17, wherein the first paa coupling step comprises coupling the first paa once and the second paa coupling step comprises coupling the second paa twice.

19. A multi-phosphorylated peptide synthesized according to the method to claim 1.

20. The multi-phosphorylated peptide of claim 19 wherein the peptide is selected from the group consisting of: SEQ ID NO. 1, SEQ ID NO. 2, SEQ ID NO. 3, SEQ ID NO. 5, SEQ ID NO. 6, SEQ ID NO. 8, SEQ ID NO. 9, SEQ ID NO. 10 and SEQ ID NO. 11.


SEQUENCE LISTING (12 sequences)

SEQ ID NO: 1 - protein, 20 aa, Artificial Sequence
  Synthetic peptide
  MISC_FEATURE (6)..(6): phosphorylation
  MISC_FEATURE (7)..(7): phosphorylation
  MISC_FEATURE (8)..(8): phosphorylation
  MISC_FEATURE (10)..(10): phosphorylation
  MISC_FEATURE (12)..(12): phosphorylation
  MISC_FEATURE (14)..(14): phosphorylation
  MISC_FEATURE (15)..(15): phosphorylation
CDDEASTTVSKTETSQVAPA

SEQ ID NO: 2 - protein, 11 aa, Artificial Sequence
  Synthetic peptide
  MISC_FEATURE (1)..(1): phosphorylation
  MISC_FEATURE (3)..(3): phosphorylation
  MISC_FEATURE (5)..(5): phosphorylation
  MISC_FEATURE (6)..(6): phosphorylation
SKTETSQVAPA

SEQ ID NO: 3 - protein, 19 aa, Artificial Sequence
  Synthetic peptide
  MISC_FEATURE (5)..(5): phosphorylation
  MISC_FEATURE (6)..(6): phosphorylation
  MISC_FEATURE (7)..(7): phosphorylation
  MISC_FEATURE (9)..(9): phosphorylation
  MISC_FEATURE (11)..(11): phosphorylation
  MISC_FEATURE (13)..(13): phosphorylation
  MISC_FEATURE (14)..(14): phosphorylation
DDEASTTVSKTETSQVAPA

SEQ ID NO: 4 - protein, 17 aa, Artificial Sequence
  Synthetic peptide
  MISC_FEATURE (9)..(9): phosphorylation
DDEASTTVSKTETSQVA

SEQ ID NO: 5 - protein, 19 aa, Artificial Sequence
  Synthetic peptide
  MISC_FEATURE (6)..(6): phosphorylation
  MISC_FEATURE (7)..(7): phosphorylation
  MISC_FEATURE (9)..(9): phosphorylation
DDEASTTVSKTETSQVAPA

SEQ ID NO: 6 - protein, 20 aa, Artificial Sequence
  Synthetic peptide
  MISC_FEATURE (7)..(7): phosphorylation
  MISC_FEATURE (8)..(8): phosphorylation
  MISC_FEATURE (15)..(15): phosphorylation
CDDEASTTVSKTETSQVAPA

SEQ ID NO: 7 - protein, 19 aa, Artificial Sequence
  Synthetic peptide
  MISC_FEATURE (5)..(5): phosphorylation
  MISC_FEATURE (7)..(7): phosphorylation
  MISC_FEATURE (11)..(11): phosphorylation
  MISC_FEATURE (14)..(14): phosphorylation
DDEASTTVSKTETSQVAPA

SEQ ID NO: 8 - protein, 16 aa, Artificial Sequence
  Synthetic peptide
  MISC_FEATURE (3)..(3): phosphorylation
  MISC_FEATURE (4)..(4): phosphorylation
  MISC_FEATURE (6)..(6): phosphorylation
  MISC_FEATURE (8)..(8): phosphorylation
  MISC_FEATURE (10)..(10): phosphorylation
ASTTVSKTETSQVAPA

SEQ ID NO: 9 - protein, 20 aa, Artificial Sequence
  Synthetic peptide
  MISC_FEATURE (6)..(6): phosphorylation
  MISC_FEATURE (7)..(7): phosphorylation
  MISC_FEATURE (8)..(8): phosphorylation
  MISC_FEATURE (10)..(10): phosphorylation
  MISC_FEATURE (12)..(12): phosphorylation
CDDEASTTVSKTETSQVAPA

SEQ ID NO: 10 - protein, 20 aa, Artificial Sequence
  Synthetic peptide
  MISC_FEATURE (7)..(7): phosphorylation
  MISC_FEATURE (8)..(8): phosphorylation
  MISC_FEATURE (10)..(10): phosphorylation
  MISC_FEATURE (12)..(12): phosphorylation
  MISC_FEATURE (14)..(14): phosphorylation
  MISC_FEATURE (15)..(15): phosphorylation
CDDEASTTVSKTETSQVAPA

SEQ ID NO: 11 - protein, 14 aa, Artificial Sequence
  Synthetic peptide
  MISC_FEATURE (1)..(1): phosphorylation
  MISC_FEATURE (2)..(2): phosphorylation
  MISC_FEATURE (4)..(4): phosphorylation
  MISC_FEATURE (6)..(6): phosphorylation
  MISC_FEATURE (8)..(8): phosphorylation
  MISC_FEATURE (9)..(9): phosphorylation
TTVSKTETSQVAPA

SEQ ID NO: 12 - protein, 9 aa, Artificial Sequence
  Synthetic peptide
  MISC_FEATURE (1)..(1): phosphorylation
  MISC_FEATURE (3)..(3): phosphorylation
  MISC_FEATURE (4)..(4): phosphorylation
TETSQVAPA